(19) United States
(12) Patent Application Publication
(10) Pub. No.: US 2025/0382672 A1
(43) Pub. Date: Dec. 18, 2025

(54) METHOD OF DETECTING CLONAL HAEMATOPOIESIS OR CANCER OR PERFORMING ANTENATAL SCREENING AND KITS

(71) Applicants: The Chancellor, Masters and Scholars of the University of Oxford, Oxford (GB); The University Court of The University of Edinburgh, Edinburgh (GB)

(72) Inventors: Bethan PSAILA, Oxford (GB); Adam MEAD, Oxford (GB); Christopher GREGORY, Edinburgh (GB)

(21) Appl. No.: 18/848,378

(22) PCT Filed: Mar. 21, 2023
(86) PCT No.: PCT/US2023/050714

§ 371 Date: Sep. 18, 2024

(30) Foreign Application Priority Data
Mar. 21, 2022 (GB) 2203947.3

(51) Int. Cl. C12Q 1/6886

(52) U.S. Cl. CPC C12Q 1/6886; C12Q 2600/156

(57) ABSTRACT

The present invention relates to the detection of disease states such as clonal haematopoiesis and cancer by analysing thrombocytes for disease associated markers. The methods comprises steps of providing a biological sample comprising thrombocytes;
extracting nucleic acid from said biological sample;
analysing said nucleic acid to identify the presence of one or more clonal haematopoiesis associated mutations or cancer associated nucleic acid fragments; and
indicating the presence or prognosis of clonal haematopoiesis or cancer based on the presence of one or more clonal haematopoiesis associated mutations or cancer associated nucleic acid fragments.

### FIELD OF THE INVENTION

The present invention relates to the detection of disease states such as clonal haematopoiesis and cancer by analysing thrombocytes for disease associated markers.

### BACKGROUND

All blood cells derive from haematopoietic stem cells (HSC) which are multipotent and self-renewing progenitor cells. HSC generate lymphoid and myeloid progenitors. The latter can differentiate into granulocytes, monocytes, red blood cells and megakaryocytes. Platelets (also called thrombocytes) are ‘blood cell fragments’ produced by megakaryocytes and are the second most abundant cell type in circulation in peripheral blood. They are packaged with a rich protein cargo, and although they do not contain a nucleus, they receive a diverse repertoire of RNA molecules from ‘parent’ megakaryocytes and contain all the necessary machinery to process and translate this for protein synthesis. Platelets also have a high capacity for intracellular trafficking and endocytosis. During peripheral circulation, they actively internalize and decipher biomolecules encountered—including proteins and nucleic acids released during tissue damage or by viral pathogens—enabling them to interpret and respond to signals in their immediate environment. Therefore, in addition to their crucial role in blood clotting and vascular maintenance, platelets function as key players in innate immunity and tumour immunosurveillance—acting as ‘vascular vacuum cleaners’ and sensing tissue damage, transformation and infection.

Therefore, analysis of platelets may be used for the detection of certain diseases such as clonal haematopoiesis (CH) and cancer.

CH develops when a haematopoietic stem cell starts making cells with the same genetic mutation, leading to an over-representation of a single clone of blood cells. CH is common in aged populations, is a pre-cancerous state, detectable by analysis of granulocyte DNA in >10% of persons aged >70 years and increases the risk of development of blood cancer by ˜10-fold and a ˜2-fold increase in cardiovascular disease, a similar increase in risk of venous thromboembolism with a significant increase in all-cause mortality. Studies have also associated CH with a range of other disorders, including degenerative, diabetes and autoimmune diseases. The degree of risk depends on the specific mutant allele driving clonal expansion, number of mutations, mutant allele burden and concomitant nongenetic risk factors such as hypertension or cigarette smoking.

Identification of persons with CH is important for early detection, and intervention could reduce the risk of complications. The cardiovascular risk associated with CH is of greater consequence than relatively rare neoplastic progression. An anti-inflammatory approach may be helpful in preventing cardiac events and also led to fewer incident lung cancers (e.g. CANTOS trial, see “Product type, therapeutic area and indication(s)” section for more info). Currently, the presence of CH is based on the identification of clones present at a frequency of 2%, which is based on the lower limit of detection of the majority of commonly used assays.

Further, platelets contain mRNA transcripts and active splicing machinery, mostly derived from their parent megakaryocytes although they can pick up and carry nucleic acids that originate from tumour cells. Other groups have focused on studying the gene expression signatures in the platelet transcriptome. However, the platelet transcriptome alone is likely to be poorly specific for cancer and hard to distinguish from non-malignant inflammatory conditions. Presently, the main approach for liquid biopsy is the analysis of tumour cell derived, cell free DNA (cfDNA) where the major issue is low abundance of cfDNA leading to low sensitivity, especially for early-stage tumours. Therefore new methods that increase the availability of tumour cell derived cfDNA for analysis via liquid biopsy approaches are needed.

As such there is a need to develop further methods for the detection of CH and cancer.

### SUMMARY OF THE INVENTION

Platelets are small (2-5 μm), multi-functional cells that originate from megakaryocytes in the bone marrow and lung. Although platelets are anucleate, they contain RNAs derived from parent megakaryocytes and the necessary translational machinery for protein synthesis. During cell death and aberrant mitosis, nucleated cells release chromosomal DNA that is rapidly fragmented resulting in ‘cell free’ DNA in plasma (cfDNA). An excess of cfDNA is deleterious. Given their ability to sense and internalize pathogen-derived nucleic acids, the present inventors hypothesized that platelets may play a role in the clearance of endogenous cfDNA. Here we reveal that despite the absence of a nucleus, platelets contain a repertoire of DNA fragments that map across the nuclear genome, in addition to mitochondrial DNA. The inventors show that this DNA is acquired from non-megakaryocyte lineage cells, demonstrating the presence of fetal DNA in maternal platelets and cancer cell-derived DNA in platelets from patients with pre-malignant lesions and overt solid cancers. This study establishes a role for platelets in the sequestration of cfDNA, an aspect of platelet biology that has not previously been highlighted, with broad applicability for minimally-invasive liquid biopsy. As platelets are easily isolated and continuously circulate through tissues, they are ideal ‘sentinels’ for genetic perturbations

Platelets are fundamental to haemostasis and vascular maintenance, and contribute to innate and adaptive immunity, including by triggering inflammatory responses via sensing of pathogen-derived nucleic acids. As part of anti-viral immunity, platelets internalize DNA and RNA viruses and, intriguingly, it was recently reported that nucleic acids derived from pine tree pollen were detectable within human platelets, indicating that platelets sequester exogenous nucleic acids encountered during circulation. While platelet RNA is well studied and has emerging utility as a liquid biopsy approach for haematological and solid malignancies, whether platelets contain DNA and, if so, its cellular origin, has not been extensively investigated.

Analysis of cfDNA in plasma is rapidly being implemented in a wide range of clinical settings, including cancer care pathways and prenatal genetic testing. A major obstacle to the utility of cfDNA for cancer surveillance is the low abundance of circulating tumour-derived DNA (ctDNA) in standard cfDNA preparations, which involve isolation of DNA from platelet-depleted plasma. Recent efforts to overcome this have focused on increasing sequencing depth or more broadly capturing cancer-associated genetic aberrations via whole genome sequencing (WGS) or epigenetic analysis. However, improved pre-analytical methods that increase capture of cfDNA would be highly beneficial in many diagnostic settings. Given their role in the sensing of pathogen-derived nucleic acids, we hypothesized that platelets may clear cfDNA from plasma, and that important insights may be derived from the analysis of genetic material in platelets that derives from cell types encountered during their peripheral circulation.

The present inventors have developed methods for the isolation of platelets from the blood, and for the subsequent extraction of the nucleic acids, RNA and DNA, from these platelets. The groups have demonstrated the identification of disease-associated gene mutations in the isolated nucleic acids. Analysis of patient samples shows that mutations are often detectable in the platelets from patients which are not detectable in other blood components. As such, the present methods significantly increase the sensitivity of mutation detection. Using these approaches, the inventors have demonstrated the utility of the analyses of platelet-derived nucleic acids in the detection of pre-malignant blood disorders, haematological cancers and solid tumours.

A first aspect of the invention relates to a method for the detection or prognosis of clonal haematopoiesis comprising:
providing a biological sample comprising thrombocytes;
extracting nucleic acid from said biological sample;
analysing said nucleic acid to identify the presence of one or more clonal haematopoiesis associated mutations; and
indicating the presence or prognosis of clonal haematopoiesis based on the presence of one or more clonal haematopoiesis associated mutations.

An aspect of the invention relates to a method for the detection or prognosis of cancer comprising:
providing a biological sample comprising thrombocytes;
extracting nucleic acid from said biological sample;
analysing said nucleic acid to identify the presence of one or more cancer associated nucleic acid fragments, in certain embodiments including cancer associated mutations and methylation profiles; and
indicating the presence or prognosis of cancer based on the presence of cancer associated nucleic acid fragments.

An aspect of the invention relates to a method of determining a treatment for a subject, comprising:
the method of detection or prognosis as described herein; and
determining a suitable treatment.

An aspect of the invention relates to a kit comprising reagents for the extraction of nucleic acid from platelets and a panel of reagents that specifically bind to and/or amplify one or more clonal haematopoiesis associated mutation(s), and optionally instructions for use.

An aspect of the invention relates to a kit comprising reagents for the extraction of nucleic acid from platelets and a panel of reagents that specifically bind to and/or amplify one or more cancer associated modifications, or cancer specific mutations.

An aspect of the invention relates to a method of treatment of a subject with cancer comprising the steps of:
providing a biological sample comprising thrombocytes,
extracting nucleic acid from said biological sample,
analysing said nucleic acid to identify the presence of one or more cancer associated nucleic acid fragments,
selecting a treatment; and
administering the treatment.

An aspect of the invention relates to a method of preparing a nucleic acid fraction comprising the steps of:
providing a biological sample comprising thrombocytes,
extracting nucleic acid from said biological sample to form a nucleic acid sample,
enriching said nucleic acid sample for one or more cancer associated nucleic acid
fragments and/or clonal haematopoiesis associated fragments.

An aspect of the invention relates to a method of genetically typing a sample of thrombocytes comprising:
providing a biological sample comprising thrombocytes;
extracting RNA from said biological sample;
converting RNA to cDNA;
analysing said cDNA to identify the presence of one or more clonal haematopoiesis associated mutations, thereby genetically typing the sample.

An aspect of the invention relates to a method of genetically typing a sample of thrombocytes comprising:
providing a biological sample comprising thrombocytes;
extracting nucleic acid from said biological sample;
analysing said nucleic acid to identify the presence of one or more cancer associated nucleic acid fragments, thereby genetically typing the sample.

An aspect of the invention relates to a method for antenatal screening for foetal genetic information, comprising the steps of:
providing a biological sample comprising thrombocytes, obtained from a pregnant woman;
extracting nucleic acid from said biological sample;
analysing said nucleic acid for genetic information related to the foetus.

### FIGURES

FIG. 1: Overview of platelet and cfDNA isolation and purity assessment. a, (i) Method for simultaneous extraction of DNA from platelet pellet (pDNA) and platelet-depleted plasma (cfDNA) from peripheral blood; (ii) method for platelet isolation and cryopreservation. Platelet freezing medium (0.9% (w/v) NaCl with 27% DMSO, diluted in the platelet-rich plasma to a final DMSO concentration of 6%). b, Platelet numbers following cryopreservation. Absolute number of platelets in whole blood (WB), platelet rich plasma (PRP) and following cryopreservation and freezing as assessed using an automated cell counter. c, Flow cytometry analysis of the platelet pellet confirming high yield of platelets with minimal leukocyte and red blood cell contamination. Analysis of the platelet pellet shown in leukocyte (left) and platelet (right) size scatter windows using a threshold of 5 000 to capture leukocytes and red cells and a lower threshold of 1 000 to capture platelets. Percentages of contaminating cells in total events shown. d, Gene expression analysis of platelet pellet shows minimal expression of red blood cell (RBC) and white blood cell (WBC)-associated genes. e, Cytospin with Wright-Giemsa stain and quantification of leukocyte contamination in platelet pellet for a representative sample. FIG. 1A & B created with BioRender.com. CH, clonal haematopoiesis; ddPCR, droplet digital PCR; DMSO, dimethyl sulfoxide; NGS, next-generation sequencing; PLT, platelet; PRP, platelet-rich plasma; RBC, red blood cell; WB, whole blood; WBC, white blood cell.

FIG. 2: Detection of platelet-biased JAK2V617F-related clonal haematopoiesis. a, JAK2V617F ddPCR assay accuracy studies. Measurement error studies showed that both (gDNA and cDNA) assays were both accurate and precise. (i) Results of reproducibility studies for the cDNA assay for serial dilutions down to a final expected fractional abundance of 0.1%. Reproducibility was assessed in three independent ddPCR runs. Repeatability studies for different (ii) gDNA and (ii) cDNA input (down to 2.5 ng and 10 ng, respectively) were conducted to assess performance of the assays for low DNA input. b, JAK2V617F ddPCR assay QC. The ddPCR assay was optimized using experimental controls consisting of cell line derived DNA (left) and granulocyte DNA from patients (right), confirming highly specific detection of mutant JAK. Lower plots-ddPCR analysis of two representative donors showing JAK2V617F-CH detected exclusively in platelets (PLT cDNA) and not in granulocytes (GRA gDNA). Red boxes highlight positive mutant FAM signal. (c) Analysis of 151 donors suggests that the study of platelets substantially increases sensitivity of detection of CH. The bar plot (left panel) shows the number of cases of JAK2V617F-related CH detected in the cohort and their distribution amongst GRA gDNA and PLT cDNA sample products. Shaded bars show cases that meet the criteria for CHIP, with a fractional abundance equal or more than 2%. The scatter plot (right panel) shows the comparison of the JAK2V6167F fractional abundance in granulocyte-derived gDNA and the platelet-derived cDNA for the total number of CH, revealing significantly higher fractional abundance for platelet-cDNA (Mann-Whitney test; p=0.0383). Bar and error bars show median and 95% CI, respectively. The red grid line corresponds to fractional abundance 2%. CH, clonal haematpoiesis, CHIP, clonal haematopoiesis of indeterminate significance; ddH2O, double-distilled water; ddPCR, droplet digital PCR; Ctrl, control; GRA, granulocyte; MNC, mononuclear cell; PLT, platelet.

FIG. 3: Detection of platelet-biased clonal haematopoiesis in genes other than JAK2. a, Custom-made, hybridisation capture panel for parallel sequencing of gDNA and cDNA for detection of the common CH-associated gene mutations. b, Schematic representation of the panel design, showing probes for JAK2. Exonic regions are shown in blue. The target regions (being exons 12 and 14) are shown in green. Given that exon 12 is 128 bp long, a single 120-nucleotide-long, centrally-aligned probe (shown in yellow) was designed to serve variant detection for both gDNA and cDNA. However, regarding exon 14, which is 88 bp, a 120-nucleotide-long probe (shown in grey), expanding to the flanking intronic regions, was designed for gDNA capture, and an additional exon-exon probe (shown in orange) involving exon 14 and part of exon 15 was designed for cDNA capture, allowing the simultaneous analysis of gDNA and cDNA templates. c, Oncoplots showing NGS-detected variants with a VAF over 2% per gene (each row=gene) in (i) GRA gDNA and (ii) PLT cDNA for each individual (each column=separate donor). CI, confidence interval; GRA, granulocyte; NGS, next-generation sequencing; PLT, platelet.

FIG. 4: Detection of JAK2V617F in pDNA, cfDNA and paired granulocyte genomic DNA. a, Results of ddPCR analysis for the detection and quantification of JAK2V617F in genomic DNA extracted from platelets, plasma and granulocytes from a representative patient diagnosed with a JAK2-mutant myeloproliferative neoplasm. FAM channel-positive events on the y axis correspond to JAK2V617F positivity, and HEX channel-positive events on the x axis correspond to JAK2 WT events. b, Analysis of five donors showing variant allele frequency of JAK2V617F in genomic DNA extracted from platelets, plasma and granulocytes.

FIG. 5. Platelets sequester DNA during circulation. a, Abundance of DNA in cfDNA and pDNA calculated as genomic equivalents per microliter (μl) of plasma. Chart shows median±95% Cl, ****p<0.0001 for Wilcoxon paired signed rank test for pDNA vs cfDNA for non-cancer controls (n=14), patients with pre-malignant, colonic sessile serrated lesions (SSL, n=29), and patients with known adenocarcinoma (n=21). b, Representative electrophoresis of DNA extracted from cfDNA and pDNA, with some platelet samples showing peaks at ˜150 base pairs (bp) and nucleosomal footprints in pDNA, similar to cfDNA. All platelet samples show larger DNA fragments ranging from ˜12′000-16′000 bp are also observed in pDNA but not in cfDNA. c, Impact of DNase treatment on the detection of JAK2 mutant alleles in pDNA and cfDNA. Data represents 3 independent experiments, mean±SD. ns p>0.05 and **p<0.01 for Wilcoxon paired signed rank test. d, Healthy donor human platelets identified by CD42-488 (cyan) show an internal fluorescent signal for NUCLEAR-ID, a cell permeable dye that intercalates with double stranded DNA (magenta). White boxes show magnified regions, scale bars represent 2 μm. e, timecourse of DNA uptake by platelets.

FIG. 6: Extracellular DNA fragments bearing cancer-associated gene mutations are sequestered by platelets. a, Number of mutant alleles per μl of pDNA extracted from platelets incubated with (+) or without (−) colorectal (LS180, COLO205 and HCT116) and erythroleukemia (HEL) cell lines. Mean±SD, n=3 independent experiments, *p<0.05 calculated using Wilcoxon paired signed rank test. b, Representative droplet digital PCR analysis showing quantification of wild-type BRAF and BRAFV600E alleles in pDNA isolated from healthy donor platelets before (top) and after (lower) co-incubation with BRAFV600E positive COLO205 colorectal cancer cells.

FIG. 7: Platelets contain a repertoire of DNA fragments that map over the human nuclear genome including tumour-derived DNA in patients with active malignancy. a, Chromosome mapping of s-pDNA and paired cfDNA for a representative patient with pancreatic adenocarcinoma (sample 6). b, Percentage of fragments mapping to the mitochondrial genome from cell free DNA (cfDNA), short (>100 and <600 bp) and long (>600 bp) platelet DNA (pDNA) fragments (s-pDNA and I-pDNA). Median value±individual data points shown. c, Deviation from median coverage in 100 kilobase (kb) windows across all chromosomes for cfDNA (top) and s-pDNA (bottom) for sample 6, revealing chromosomal aberrations in chromosomes 2, 6 and 7 (copy number gains and amplifications shown in red, deletions in green). d, Read depth distribution around transcription start sites of genes highly (TPM>15, purple) or lowly (TPM<=15, blue) expressed in peripheral blood mononuclear cells (PBMCs) for cfDNA (top) and s-pDNA (bottom) mononucleosome reads. Depth per position per sample was normalised to the median read depth across all genes in that expression category. MNCs, mononuclear cells; pos, position; TSS, transcriptional start site.

FIG. 8: KRASG12D copies are more abundant in pDNA than cfDNA in mice with localized and metastatic colorectal adenocarcinoma. a, Waterfall plot showing fold difference in copies of KRASG12D detected per ul of DNA for pDNA vs. cfDNA. b & c, Representative droplet digital PCR plots showing KRASG12D (blue) in higher abundance in pDNA than in cfDNA in two representative mice, b, a KPN mouse and c, a KP mouse. CRC, colorectal cancer; WT, wild-type.

FIG. 9: BRAFV600E in platelets from patients with pre-malignant lesions detected on colonoscopy screening. a, Pie charts showing detection of BRAFV600E in patients with high-risk, premalignant colonic lesions (sessile serrated lesions, SSL) (n=29) and control individuals undergoing colonoscopy screening in whom no lesion or malignancy was detected (n=14). Mutant BRAF was detected in 17.2% (5/29) of SSL patients and 0% (0/14) of controls. b,

Relative copy number of BRAFV600E in pDNA compared to cfDNA in the 5 patients with SSLs in whom mutant BRAF was detectable in either cfDNA or pDNA. Data were converted to a base-2 logarithm with a pseudo count of 1. c, Droplet digital PCR plot showing BRAFV600E (blue) in pDNA (top) and cfDNA (lower plot) in two SSL patients. SSL, sessile serrated lesions; WT, wild-type.

FIG. 10: Proof-of-principle utility of pDNA analysis for antenatal genetic screening. a, Fluorescence in-situ hybridization chromosome paint and droplet digital PCR (ddPCR) showing detection of the Y-chromosome gene SRY in platelets of pregnant mothers carrying male babies, but not in mononuclear cells (MNCs). Mother's blood was sampled prior to delivery. Platelets and MNCs were counterstained with β-tubulin (blue) and imaged using a ZEISS LSM900, 63× magnification. Representative images shown. b, Detection of SRY gene copies by ddPCR of DNA isolated from maternal platelets 24 and 48 hours after caesarean section delivery of a male infant (at delivery: n=9, 24 hours after delivery: n=5 and 48 hours after delivery: n=2). Mean ±SD shown.

FIG. 11: Platelets sequester DNA during circulation. a, Healthy donor human platelets identified by CD42-488 (cyan) show an internal fluorescent signal for NUCLEAR-ID, a cell permeable dye that intercalates with double stranded DNA (magenta). White boxes show magnified regions, scale bars represent 2 μm. b, Method for simultaneous extraction of DNA from platelet pellet (pDNA) and platelet-depleted plasma (cfDNA) from peripheral blood. c, Abundance of DNA in cfDNA and pDNA calculated as genomic equivalents per microliter (ul) of plasma. Chart shows median±95% CI, ****p<0.0001 for Wilcoxon paired signed rank test for pDNA vs cfDNA for non-cancer controls (n=14), patients with pre-malignant, colonic sessile serrated lesions (SSL, n=29), and patients with known adenocarcinoma (n=21). d, Fluorescence in-situ hybridization chromosome paint and droplet digital PCR (ddPCR) showing detection of the Y-chromosome gene SRY in maternal platelets but not mononuclear cells (MNCs) sampled from mothers of male neonates prior to delivery. Platelets and MNCs were counterstained with β-tubulin (blue) and imaged using a ZEISS LSM900, 63× magnification. Representative images shown. e, Detection of SRY gene copies by ddPCR of DNA isolated from maternal platelets 24 and 48 hours after caesarean section delivery of a male infant (at delivery: n=9, 24 hours after delivery: n=5 and 48 hours after delivery: n=2). Mean±SD shown. f, Rise in cfDNA following acute depletion of platelets in healthy mice following administration of an anti-platelet antibody (n=20 for each of isotype control and anti-platelet antibody groups). Plots show mean (+SEM) fold change at Day 1, 3 and 5 compared to baseline (Day 0, untreated mice).

FIG. 12: Platelets rapidly internalize DNA released by nucleated cells via uptake of DNA-loaded extracellular vesicles. a, Healthy donor-derived CD42-488 (cyan)-labelled platelets before (left) and after (middle and right) co-incubation with COLO205 cells labelled with NUCLEAR-ID, to enable tracking of DNA uptake (magenta). White boxes show magnified regions, scale bars represent 2 μM. White arrows highlight platelet uptake of COLO25 DNA. Middle and right images are the same, with the bright field view shown on the right to highlight membrane of COLO205 cells. b, Live cell imaging time lapse showing internalization of fluorescently-labelled DNA by platelets. Scale bars represent 3 μm. c, Quantification (mean±SD) of platelet DNA fluorescence intensity over time (minutes). AF647 signal was also measured in the background regions of each image to detect any fluctuation in background signal. ****p<0.0001 for Wilcoxon paired signed rank test. Tracking shown for platelets incubated with control media (n=168 platelets) or media conditioned by NUCLEAR-ID-labelled COLO205 cells (n=173 platelets). d, 3D reconstruction of a cluster of extracellular vesicles (EVs) released by apoptotic BL2 cells labelled with an amine-reactive, succinimidyl ester (SE) membrane dye (CF658, red) and DNA stain (DAPI, yellow) reveals internalized DNA. Imaged using a ZEISS LSM900, 63× magnification. e, Following incubation with labelled EVs, platelets were stained with CD42-488 (cyan). EVs (red) were visualized within platelets, confirming platelet uptake of apo-EVs. 3D modelling shows 90° rotation of platelet. f, 3D reconstruction of platelet internalization of DNA (yellow)-loaded apo-EVs (red). Two representative platelets shown. g, Droplet digital PCR quantification of the Y-chromosome gene SRY in pDNA derived from platelets of a female donor after incubation with EVs derived from apoptotic BL2 cells (apo-EVs) vs. EVs from apoptotic resistant BL2 cells (non-apo EVs). h, Fluorescence in-situ hybridisation micrographs and 3D render (right) demonstrating fragments of X- and Y-chromosomes present in female donor platelets following exposure to male BL2 cells. Platelets counterstained with β-tubulin (blue). Representative images shown. Imaged using a ZEISS LSM900, 63× magnification. Images analysed using ImageJ and Imaris Viewer. Abbreviations: arbitrary intensity units (AIU); apoptotic (apo); extracellular vesicles (EVs); Burkitt's lymphoma (BL2) cells; succinimidyl ester (SE); clockwise (CW); counter-clockwise (CCW)

FIG. 13: Extracellular DNA fragments bearing cancer-associated gene mutations are sequestered by platelets and protected from degradation. a, Experimental system for co-incubation of platelets from healthy donors with malignant cells separated by 1 μm membrane inserts, enabling exchange of extracellular biomolecules and small EVs but not cells between compartments. Following co-incubation, platelets were removed and washed 3× prior to DNA extraction. Image created on BioRender.com b, Number of mutant alleles per ul of pDNA extracted from platelets incubated with (+) or without (−) colorectal (LS180, COLO205 and HCT116) and erythroleukemia (HEL) cell lines. Mean±SD, n=3 independent experiments, *p<0.05 calculated using Wilcoxon paired signed rank test. c, Representative droplet digital PCR analysis showing quantification of wild-type BRAF and BRAFV600E alleles in pDNA isolated from healthy donor platelets before (top) and after (lower) co-incubation with BRAFV600E positive COLO205 colorectal cancer cells. d, Quantification of BRAFV600E in red blood cells (RBCs), mononuclear cells (MNCs) and platelets (PLTs) following incubation in media conditioned by COLO205 cells. Data represents three independent experiments, mean±SD. *p <0.05, using a Mann-Whitney U test. e, Impact of DNase treatment on the detection of JAK2 mutant alleles in pDNA and cfDNA. Data represents 3 independent experiments, mean±SD. ns p>0.05 and **p<0.01 for Wilcoxon paired signed rank test. Abbreviations: mononuclear cells (MNCs); platelets (PLTs); red blood cells (RBCs); wild-type (WT)

FIG. 14: Platelets contain a repertoire of DNA fragments that map over the human nuclear genome, including tumour-derived DNA in patients with active malignancy. a, Percentage of fragments mapping to the mitochondrial genome from cell free DNA (cfDNA), short (>100 and <600 bp) and long (>600 bp) platelet DNA (pDNA) fragments (s-pDNA and I-pDNA). Median value±individual data points shown. b, Plot showing significantly higher frequency of di-nucleosomal fragments (fragments above 250 bp) in s-pDNA than cfDNA (standard error of the mean is shown in grey, p<0.0005 for a chi-square test of independence). c, Fragment length distribution of paired and aligned reads for cfDNA (top) and s-pDNA samples (bottom). Samples 1-5 have a peak fragment length of around 165 bp, representing mono-nucleosomal fragments, with a second smaller peak around 325 bp (shown in inset plot), representing di-nucleosomal fragments. The mono- and di-nucleosomal fragment lengths from sample 6 (red line), The mono- and di-nucleosomal fragment lengths from sample 6 (red line), a patient with untreated pancreatic adenocarcinoma, are notably shorter than samples 1-5 (from patients following anti-cancer therapy). d, Deviation from median coverage in 100 kilobase (kb) windows across all chromosomes for cfDNA (top) and s-pDNA (bottom) for sample 6, revealing chromosomal aberrations in chromosomes 2, 6 and 7 (copy number gains and amplifications shown in red, deletions in green). e, Read depth distribution around transcription start sites of genes highly (TPM>15, purple) or lowly (TPM<=15, blue) expressed in peripheral blood mononuclear cells (PBMCs) for cfDNA (top) and s-pDNA (bottom) mononucleosome reads. Depth per position per sample was normalised to the median read depth across all genes in that expression category. Abbreviations: peripheral blood mononuclear cells (PBMNCs); position (pos); transcriptional start site (TSS).

FIG. 15: KRASG12D copies are more abundant in pDNA than cfDNA in mice with localized and metastatic colorectal adenocarcinoma. a, Schematic showing isolation of platelet DNA (pDNA) and cell free (cfDNA) from C57BL/6 mice expressing KRASG12D and TP53 (KP) mutations via the villin promotor resulting in locally-invasive colorectal adenocarcinoma, and mice with KRASG12D, TP53 and NOTCH (KPN) mutations with aggressive, metastatic disease. Image created with BioRender.com. b, Waterfall plot showing fold difference in copies of KRASG12D detected per ul of DNA for pDNA vs. cfDNA. c & d, Representative droplet digital PCR plots showing KRASG12D (blue) in higher abundance in pDNA than in cfDNA in two representative mice, c, a KPN mouse and d, a KP mouse. Abbreviations: colorectal cancer (CRC); wild-type (WT).

FIG. 16: BRAFV600E in platelets from patients with pre-malignant lesions detected on colonoscopy screening. a, Pie charts showing detection of BRAFV600E in patients with high-risk, premalignant colonic lesions (sessile serrated lesions, SSL) (n=29) and control individuals undergoing colonoscopy screening in whom no lesion or malignancy was detected (n=14). Mutant BRAF was detected in 17.2% (5/29) of SSL patients and 0% (0/14) of controls. b, Relative copy number of BRAFV600E in pDNA compared to cfDNA in the 5 patients with SSLs in whom mutant BRAF was detectable in either cfDNA or pDNA. Data were converted to a base-2 logarithm with a pseudo count of 1. c, Droplet digital PCR plot showing BRAFV600E (blue) in pDNA (top) and cfDNA (lower plot) in two SSL patients. Abbreviations: sessile serrated lesions (SSL); wild-type (WT).

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

The present invention is based on the finding that mutations associated with clonal haematopoiesis (CH) can be detected in anucleated cells such as thrombocytes. The standard method for detecting CH is by analysis of whole blood or white blood cells. Thrombocytes are not typically analysed by standard approaches applied for the detection of cancer associated gene mutation, but also accumulating evidence demonstrates that many long-term haematopoietic stem cells (HSCs) produce cells exclusively of the platelet lineage. All CH studies to date are based on whole blood or granulocyte DNA and therefore 1) they do not assess for platelet-restricted clones, and 2) the amount of ribonucleic acid contained in platelets would be diluted and the mutations missed by sequencing nucleic acids from whole blood samples. However, the present inventors have demonstrated that analysis of nucleic acids specifically from isolated thrombocytes is much more sensitive than previous methods which analyse whole blood or white blood cells.

As such, an aspect the invention relates to a method for the detection or prognosis of haematopoiesis comprising:
providing a biological sample comprising thrombocytes;
extracting nucleic acid from said biological sample;
analysing said nucleic acid to identify the presence of one or more clonal haematopoiesis associated mutations; and
indicating the presence or prognosis of haematopoiesis based on the presence of one or more clonal haematopoiesis associated mutations.

Clonal haematopoiesis (CH) occurs when a haematopoietic stem cell begins to make cells with the same genetic mutation. CH is a condition where blood cancer-associated mutations are detectable in the blood cells of people with normal blood cell parameters. This condition is common in individuals over 70 years of age (detectable in >10%). CH also increases risks of blood cancer as well as cardiovascular disease. CH may be considered a pre-disease state i.e. a state wherein patients are identified as being at risk of developing further disease state. The detection of CH is important as it can be used as a biomarker for early detection of blood cancers, as well as the risk of blood clots and cardiovascular disease, opening up opportunities for preventative interventions. As such detection of CH can lead to detection of subjects at high risk of developing blood cancers, blood clots and cardiovascular disease.

As used herein the term “platelets” and “thrombocytes” are used interchangeably to refer to anucleate ‘blood cell fragments’ that are produced by bone marrow megakaryocytes. Platelets are the second most abundant cell in circulation in peripheral blood and have a primary role in the prevention of bleeding and maintaining homeostasis. Platelets do not have a nucleus, however they are packaged with RNA molecules by ‘parent’ megakaryocytes, which they are able to translate for protein synthesis.

The method of the present invention involves extracting nucleic acid from thrombocytes which is subsequently analysed for the presence of CH associated mutations. The nucleic acid that is extracted may be RNA or DNA, or RNA and DNA extracted simultaneously. RNA may be extracted from the biological sample without parallel extraction of DNA. DNA may be extracted from the biological sample without parallel extraction of RNA. Where RNA is extracted from thrombocytes, the RNA is subsequently converted to cDNA for analysis. The DNA that is extracted from the thrombocytes may be genomic DNA (gDNA). Where a combination of DNA and RNA are extracted from the thrombocytes the method may comprise parallel analysis of cDNA and gDNA. The analysis of cDNA and gDNA may be performed simultaneously, sequentially or separately to detect CH associated mutations. In one embodiment RNA and gDNA are extracted from thrombocytes and analysed in parallel for the presence of CH associated mutations. In one embodiment RNA and gDNA are extracted from thrombocytes and analysed separately for the presence of CH associated mutations. In one embodiment RNA is extracted from thrombocytes and analysed for the presence of CH associated mutations. In one embodiment gDNA is extracted from thrombocytes and analysed for the presence of CH associated mutations.

The conversion of RNA to cDNA may be performed using any suitable method known in the art, for example the extracted RNA is converted to cDNA via reverse transcription. A reverse transcriptase enzyme can be used to convert RNA to cDNA. Reverse transcriptase, also known as RNA-dependent DNA polymerase, is an enzyme used to generate complementary DNA (cDNA) from an RNA template. Specifically, the enzyme is a DNA polymerase enzyme that transcribes single-stranded RNA into DNA. This enzyme is able to synthesize a double helix DNA once the RNA has been reverse transcribed in a first step into a single-strand DNA. RNA can be reverse transcribed into cDNA using RNA-dependent DNA polymerases such as, for example, reverse transcriptases from viruses, retrotransposons, bacteria, etc. These can have RNase H activity, or reverse transcriptases can be used that are so mutated that the RNase H activity of the reverse transcriptase was restricted or is not present (e.g. MMLV-RT RNase H). Suitable reverse transcriptases include but are not limited to: AMV reverse transcriptase, MMLV reverse transcriptase, engineered MMLV reverse transcriptase. RNA-dependent DNA synthesis (reverse transcription) can also be carried by enzymes that show altered nucleic acid dependency through mutation or modified reaction conditions and thus obtain the function of the RNA-dependent DNA polymerase. Commercial kits are available to reverse transcribe RNA into cDNA. Once the RNA is reverse transcribed into cDNA, the DNA sequence can be analysed for the presence of specific mutations or expression profiles associated with disease states. Expression profiles may be determined using selective nucleic acid hybridization as described above. Such techniques are well known in the art and may comprise selective amplification using amplification primers that are specific for the mutation to be detected or selective hybridization to nucleic acid arrays using mRNA-specific probes. Alternatively, general primers can be used to amplify the DNA comprising the suspected mutation and the mutation can then be detected in the amplicon by selective nucleic acid hybridization using probes that are specific for the mutation

The term “clonal haematopoiesis associated mutations” refers to any mutation that is indicative of CH. Mutations which are indicative or associated with CH may be identified by comparing samples obtained from subject known to have CH with samples obtained from healthy subjects. CH associated mutations are those which are found within diseased samples. The present method may detect one or more, two or more, three or more, four or more, five or more, or ten or more CH associated mutations. For example, the method may comprise detecting a panel of CH associated mutations. In an embodiment the CH associated mutations may be present in one or more of the following genes; JAK2 (Ensembl ID: ENSG00000096968), CALR (Ensembl ID: ENSG00000179218), MPL (Ensembl ID: ENSG00000117400), CBL (Ensembl ID: ENSG00000110395), KRAS (Ensembl ID: ENSG00000133703), GNB1 (Ensembl ID: ENSG00000078369), DNMT3A (Ensembl ID: ENSG00000119772), TET2 (Ensembl ID: ENSG00000168769), ASXL1 (Ensembl ID: ENSG00000171456), IDH2 (Ensembl ID: ENSG00000182054), SF3B1 (Ensembl ID: ENSG00000115524), SRSF2 (Ensembl ID: ENSG00000161547), U2AF1 (Ensembl ID: ENSG00000160201), PPM1D (Ensembl ID: ENSG00000170836), TP53 (Ensembl ID: ENSG00000141510). In an embodiment the one or more CH associated mutations are selected from JAK2V^{617F}, JAK2 exon12, CALR exon9, MPL^{S5050}, MPL^{W515}, CBL exon 8, CBL exon 9, KRAS exon 2, KRAS exon 3, GNB1 exon 5, DNMT3A all exons, TET2 all exons, ASXL1 exon 12, IDH2 exon 4, SF3B1 exon 14, SF3B1 exon 15, SRSF2^{P95}, U2AF1 exon 2, U2AF1 exon 6, PPM1D exon 6, TP53 all exons.

The method may comprise further analysing said nucleic acid to identify the presence of one or more clonal haematopoiesis markers. These CH markers may comprise mutations or they may be other genetic aberrations or expression profiles associated with CH.

The present method may be combined with analysis of white blood cells in order to detect CH. Therefore, in an embodiment the method further comprises:
providing a biological sample comprising granulocytes;
extracting nucleic acid from said biological sample; and
analysing said nucleic acid to identify the presence of one or more clonal haematopoiesis associated mutations.

The analysis of the thrombocytes and granulocytes may be performed in parallel in the same analysis or may be performed in separate analyses. The nucleic acid extracted from the granulocytes my comprise RNA and/or DNA. Where RNA is extracted from granulocytes, the RNA which is subsequently converted to cDNA for analysis. The DNA that is extracted from the granulocytes may be genomic DNA (gDNA).

As CH may be considered a pre-disease state which indicates an increased risk of developing other disease states such as but not limited to; blood cancers, blood clots and cardiovascular disease, the method may also comprise selecting subjects identified as having CH for further monitoring. The further monitoring may comprise follow up over time to monitor the subject in order to allow early detection of subsequent development of other disease states.

In an embodiment a subject that is identified as having clonal haematopoiesis may also be identified as being at high risk of a disease selected from one or more of; cardiovascular disease, heart failure, diabetes, autoimmune disease and/or myeloid blood cancers. In particular a subject that has clonal haematopoiesis may also be identified as being at high risk of blood cancers such as myelodysplastic syndrome and acute myeloid leukaemia. Where a subject is identified as high risk of a disease state the subject may be selected for preventative treatment e.g. measures taken for the purpose of disease prevention. Preventative treatment may comprise environmental, lifestyle and/or behavioural changes that may reduce risk of the subject developing the disease state.

In an aspect the invention relates to a method of determining a treatment for a subject, comprising
the method of detection or prognosis as described herein; and
determining a suitable treatment.

In an embodiment the method of determining a treatment for a subject, comprises
the method of detection or prognosis of clonal haematopoiesis as described herein;
determining that the subject is at high risk of a disease selected from one or more of;
cardiovascular disease, heart failure, diabetes, autoimmune disease and/or myeloid blood cancers and determining a suitable treatment.

Multiple methods are known in the art which may be used to identify one or more CH associated mutations and or markers. In an embodiment the presence of one or more CH associated mutations is identified via droplet digital PCR (ddPCR), next generation sequencing, allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), pyro-sequencing, HPLC (high-performance liquid chromatography) or oligonucleotide ligation assay (OLA). In a preferred embodiment ddPCR is used to identify one or more CH associated mutations.

The present inventors have also identified that thrombocytes uptake disease specific nucleic acid fragments which can be isolated and detected from the thrombocytes. In particular thrombocytes take up cell-free DNA fragments released by solid tumour cells. The present inventors have shown that it is possible to detect tumour cell-specific gene mutations in DNA contained within thrombocytes isolated from peripheral blood. The capability to take up tumour cell-derived DNA fragments appears to be unique to platelets and does not occur with red blood cells or leukocytes. Previous platelet-based approaches for the detection of cancer have used the platelet gene expression profile to detect cancer. As platelets lack a nucleus, the platelet transcriptome is determined by (i) the mRNA ‘inherited’ from a parent megakaryocyte (ii) environmental influences on circulating platelets that alter mRNA splicing (iii) mRNA molecules that are absorbed by circulating platelets. Detection of tumour-specific gene mutations at RNA level in platelets therefore requires the mutation to be expressed at high enough levels for the mRNA to be released by tumour cells and stable enough to be transferred to circulating platelets. This approach is likely to have poor sensitivity and lack specificity to distinguish between malignant and non-malignant pathologies such as wound healing. Other approaches for the detection of cancer aim to identify cancer associated cell-free DNA from plasma samples. In contrast the present approach detects cell-free DNA fragments released by solid tumour cells, which have been taken up by circulating platelets. The present approach extracts said cell-free DNA fragments from platelets and, as demonstrated herein, allows significantly more DNA to be isolated than from the standard approach using plasma, which increases sensitivity of detection.

In an aspect the invention relates to a method for the detection or prognosis of cancer comprising:
providing a biological sample comprising thrombocytes;
extracting nucleic acid from said biological sample;
analysing said nucleic acid to identify the presence of one or more cancer associated nucleic acid fragments; and
indicating the presence or prognosis of cancer based on the presence of cancer associated nucleic acid fragments.

In an embodiment the nucleic acid that is extracted from the biological sample may be DNA and/or RNA. The nucleic acid that is extracted may be RNA or DNA, or RNA and DNA extracted simultaneously. RNA may be extracted from the biological sample without parallel extraction of DNA. DNA may be extracted from the biological sample without parallel extraction of RNA. Where RNA is extracted from thrombocytes, the RNA which is subsequently converted to cDNA for analysis. The DNA that is extracted from the thrombocytes may be genomic DNA (gDNA). Where a combination of DNA and RNA are extracted from the thrombocytes the method may comprise parallel analysis of cDNA and gDNA. The analysis of cDNA and gDNA may be performed simultaneously, sequentially or separately to detect CH associated mutations. In one embodiment RNA and gDNA are extracted from thrombocytes and analysed in parallel for the presence of cancer associated nucleic acid fragment. In one embodiment RNA and gDNA are extracted from thrombocytes and analysed separately for the presence of cancer associated nucleic acid fragments. In one embodiment RNA is extracted from thrombocytes and analysed for the presence of cancer associated nucleic acid fragments. In one embodiment gDNA is extracted from thrombocytes and analysed for the presence of cancer associated nucleic acid fragments.

The term “cancer associated nucleic acid fragment” refers to a fragment of nucleic acid that is indicative of cancer. In certain embodiments the cancer associated nucleic acid fragment is a fragment of DNA or RNA comprising a mutation which is associated with cancer. The presence of the cancer associated nucleic acid fragment indicates the presence of a mutant gene that is present in a cancer cell of the subject, wherein the cancer associated nucleic acid fragment has an altered nucleic acid sequence relative to the normal gene of a healthy control subject. The term “cancer associated nucleic acid fragment” may also refer to a nucleic acid that is produced by, expressed by, or present in a cancer cell but not in a healthy non-diseased cell. In an embodiment the term “cancer associated nucleic acid fragment” may refer to a nucleic acid that has an altered expression level (enhanced or reduced) by or in a cancer cell compared to a healthy non-diseased cell. In an embodiment the term “cancer associated nucleic acid fragment” may refer to a nucleic acid that is produced by, expressed by, or present in a normal cell but not produced by, expressed by, or present by or in a cancer cell. According to the present invention the cancer associated nucleic acid fragment is a cell-free nucleic acid fragment that has been released by a cancer and taken up by the thrombocytes. The nucleic acid fragment is not part of the platelet transcriptome but is a cell-free fragment that has been taken up by the thrombocytes. The cancer associated nucleic acid fragment may be DNA and/or RNA. In a preferred embodiment the cancer associated nucleic acid fragment is DNA. For example, the nucleic acid fragment may be a cell-free fragment of DNA released by nucleated cells, e.g. cancer cells, which has been taken up by the thrombocytes.

The skilled person will appreciate that cancer-associated nucleic acid fragments may be identified using a variety of methods and by a variety of features, for example the fragment may comprise a fragment length indicative of a DNA fragment released by cancer cells and/or a nucleosomal footprint that is typical of a DNA fragment released by cancer cells. The term “nucleosomal footprint” as used herein refers to gene expression information from the original tissue from which the fragment is derived, which is present in the nucleic acid fragment. The present inventors have shown herein that platelets uptake a variety of cell free nucleic acid fragments. Two distinct populations of nucleic acid fragment taken up by platelets have been analysed, the first population comprises longer nucleic acid fragments >10,000 base pairs (bp) and the second population comprises shorter nucleic acid fragments <600 bp. Both populations contain fragments that map to the nuclear genome however, the longer nucleic acid fragments have been shown to contain more fragments that map to mitochondrial genome and the shorter fragments have been shown to enrich for the tumour derived fraction. The cancer associated nucleic acid fragment may have a fragment length between 20 bp and 500 bp, 20 bp and 400 bp, 20 bp and 300 bp, 20 bp and 200 bp, 20 bp and 150 bp, 50 bp and 500 bp, 50 bp and 400 bp, 50 bp and 300 bp, 50 bp and 200 bp, 50 bp and 150 bp, 100 bp and 500 bp, 100 bp and 400 bp, 100 bp and 300 bp, 100 bp and 200 bp, or 100 bp and 150 bp. In a preferred embodiment the fragment length is between 50 bp and 250 bp, or 100 bp and 200 bp. In a preferred embodiment the fragment length is approximately 150 bp. In an embodiment the method for the detection or prognosis of cancer comprises a step of enriching the nucleic acid sample for shorter nucleic acid fragments, for example enriching the nucleic acid sample for fragments with a length between 20 bp and 500 bp, 20 bp and 400 bp, 20 bp and 300 bp, 20 bp and 200 bp, 20 bp and 150 bp, 50 bp and 500 bp, 50 bp and 400 bp, 50 bp and 300 bp, 50 bp and 200 bp, 50 bp and 150 bp, 100 bp and 500 bp, 100 bp and 400 bp, 100 bp and 300 bp, 100 bp and 200 bp, or 100 bp and 150 bp. In a preferred embodiment the nucleic acid sample is enriched for fragment length between 50 bp and 250 bp, or 100 bp and 200 bp.

The cancer associated nucleic acid fragment may comprise one or more markers of cancer. The markers of cancer may be a cancer associated modification, a cancer specific mutation, a cancer specific methylation pattern, a cancer specific genetic aberration and/or a cancer specific fragmentation pattern.

In an embodiment the cancer associated nucleic acid fragment is selected from nucleic fragments comprising one or more mutation that is associated with cancer. Non-limiting examples of mutations include, for example, BRAFV600E, KRASG12D, PIKCAH1047R, TP53R273H.

There are multiple methods known in the art for the detection of cancer associated nucleic acid fragments as such any suitable method may be used for the detection. In an embodiment the cancer associated nucleic acid fragment is identified via droplet digital PCR, next generation sequencing, allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), or oligonucleotide ligation assay (OLA), methylation analysis, fragmentation pattern analysis.

In an embodiment the cancer associated nucleic acid fragment may be various different sizes for example the nucleic acid fragment may comprise between 10 to 1500 nucleotides, 10 to 1400 nucleotides, 10 to 1300 nucleotides, 10 to 1200 nucleotides, 10 to 1100 nucleotides, 10 to 1000 nucleotides, 10 to 900 nucleotides, 10 to 800 nucleotides, 10 to 700 nucleotides, 10 to 600 nucleotides 10 to 500 nucleotides 10 to 400 nucleotides, 10 to 300 nucleotides, 10 to 200 nucleotides, 10 to 100 nucleotides, 50 to 1500 nucleotides, 100 to 1500 nucleotides, 200 to 1500 nucleotides, 300 to 1500 nucleotides, 400 to 1500 nucleotides, 500 to 1500 nucleotides, 600 to 1500 nucleotides, 700 to 1500 nucleotides, 800 to 1500 nucleotides, 900 to 1500 nucleotides, 1000 to 1500 nucleotides, 1100 to 1500 nucleotides, 1200 to 1500 nucleotides, 1300 to 1500 nucleotides, or 1400 to 1500 nucleotides. In an embodiment the cancer associated nucleic acid fragment comprises between 300 to 500 nucleotides, or 400 to 500 nucleotides. In an embodiment the cancer associated nucleic acid fragment comprises between 800 to 1500 nucleotides. The cancer associated nucleic acid fragment may comprise 20 nucleotides and 500 nucleotides, 20 nucleotides and 400 nucleotides, 20 nucleotides and 300 nucleotides, 20 nucleotides and 200 nucleotides, 20 nucleotides and 150 nucleotides, 50 nucleotides and 500 nucleotides, 50 nucleotides and 400 nucleotides, 50 nucleotides and 300 nucleotides, 50 nucleotides and 200 nucleotides, 50 nucleotides and 150 nucleotides, 100 nucleotides and 500 nucleotides, 100 nucleotides and 400 nucleotides, 100 nucleotides and 300 nucleotides, 100 nucleotides and 200 nucleotides, or 100 nucleotides and 150 nucleotides. In a preferred embodiment the fragment length is between 50 nucleotides and 250 nucleotides, or 100 nucleotides and 200 nucleotides. In a preferred embodiment the fragment length is approximately 150 nucleotides. Wherein multiple cancer associated nucleic acid fragments are detected the fragment may be within different size ranges. i.e., said fragments may each comprise a different number of nucleotides.

In an embodiment the method may comprise a step of separating the cancer associated nucleic acid fragment from other nucleic acid extracted from the thrombocytes based on size. The method may comprise of separating the cancer associated nucleic acid fragments based on size, wherein multiple cancer associated nucleic acid fragments of different sizes are detected in the method.

The cancer associated nucleic acid fragment may comprise DNA or RNA. Multiple nucleic acid fragments may be detected in the present methods the fragments may be DNA and/or RNA. Where a combination of DNA and RNA fragments are detected the RNA fragments may first be converted to cDNA. As such the method may comprise a step of extracting RNA from a biological sample comprising thrombocytes, converting RNA to cDNA and analysing said cDNA to identify the presence of one or more cancer associated nucleic acid fragments. Conversion of the RNA to cDNA may be performed via reverse transcription as described herein. Where a combination of DNA and RNA fragments are detected, the method may comprise parallel analysis of cDNA and gDNA. The analysis of cDNA and gDNA may be performed simultaneously, sequentially or separately to detect cancer associated nucleic acid fragments.

The cancer associated nucleic acid fragment may be associated with a solid tumour. Types of solid tumour include sarcomas, carcinomas, and lymphomas. In an embodiment the cancer associated nucleic acid fragment is associated with a cancer selected from a sarcoma, carcinoma, and/or lymphoma.

In an embodiment the cancer associated nucleic acid fragment is associated with a cancer selected from gastric cancer, lung cancer, non-small cell lung cancer, lung adenocarcinoma, lung squamous cell carcinoma, bone cancer, pancreatic cancer, colon cancer, colorectal cancer, skin cancer, cancer of the head or neck, head and neck squamous cell carcinoma, melanoma, uterine cancer, ovarian cancer, rectal cancer, cancer of the anal region, stomach cancer, testicular cancer, breast cancer, brain cancer, hepatocellular cancer, carcinoma of the fallopian tubes, carcinoma of the endometrium, carcinoma of the cervix, carcinoma of the vagina, carcinoma of the vulva, cancer of the oesophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, thymoma, urothelial carcinoma leukaemia, prostate cancer, prostatic adenocarcinoma mesothelioma, adrenocortical carcinoma, lymphomas, such as such as Hodgkin's disease, non-Hodgkin's, and multiple myelomas.

The methods described herein relate to analysing samples of thrombocytes obtained from a biological sample. The methods may be performed on any suitable body sample comprising thrombocytes, such as for instance a tissue sample comprising blood. In a preferred embodiment said sample is a blood sample for example a whole blood sample also known as a peripheral blood sample. The blood sample may be a fresh blood sample or it may be a preserved sample for example the sample may have been previously frozen or cryopreserved. Methods to obtain a blood sample or tissue sample are known in the art, for example a blood sample may be obtained via venous extraction. A tissue sample may be obtained via a biopsy.

Various steps may be applied to the biological sample in order to isolate the thrombocytes from the sample and also to enhance the extraction of the nucleic acid from said thrombocytes. Such steps are described more fully below in relation to the method of preparing a nucleic acid fraction. The biological sample may be processed to provide an isolated sample of thrombocytes.

In an aspect the invention relates to a method of determining a treatment for a subject, comprising
the method of detection or prognosis as described herein; and
determining a suitable treatment.

In an embodiment the method of determining a treatment for a subject, comprises
the method of detection or prognosis of cancer as described herein; and
determining a suitable treatment.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The suitable treatment may be selected on the basis of the mutations that are identified via the method of detection or prognosis. Wherein the method of detection or prognosis is for cancer, the suitable treatment may include a therapeutic agent or radiation therapy and includes gene therapy, viral therapy, RNA therapy bone marrow transplantation, nanotherapy, targeted anti-cancer therapies or oncolytic drugs. Examples of other therapeutic agents include checkpoint inhibitors, antineoplastic agents, immunogenic agents, attenuated cancerous cells, tumour antigens, antigen presenting cells such as dendritic cells pulsed with tumour-derived antigen or nucleic acids, immune stimulating cytokines (e.g., IL-2, IFNa2, GM-CSF), targeted small molecules and biological molecules (such as components of signal transduction pathways, e.g. modulators of tyrosine kinases and inhibitors of receptor tyrosine kinases, and agents that bind to tumour-specific antigens, including EGFR antagonists), an anti-inflammatory agent, a cytotoxic agent, a radiotoxic agent, or an immunosuppressive agent and cells transfected with a gene encoding an immune stimulating cytokine (e.g., GM-CSF), chemotherapy. In an embodiment the suitable therapy may be an immunomodulatory agent, specifically an immune checkpoint inhibitor, examples of immune checkpoint inhibitors include but are not limited to inhibitors of an immune checkpoint protein selected from the group consisting of CTLA-4, PD-1, PD-L1, PD-L2, TIM3, LAG-3, B7-H3, B7-H4, B7-H6, A2aR, BTLA, GAL9 and IDO. In an embodiment, the suitable treatment may be surgery. In an embodiment combination therapy may be used for example the combination may comprise one or more of the therapies listed herein.

The term “subject” as used herein includes, but is not limited to, mammals, including, e.g., a human, a non-human primate, a mouse, a pig, a cow, a goat, a cat, a rabbit, a rat, a guinea pig, a hamster, a degu, a horse, a monkey, a sheep, or other non-human mammal; and non-mammal animals, including, e.g., a non-mammalian vertebrate, such as a bird (e.g., a chicken or duck) or a fish, and an invertebrate. The subject may be a healthy animal or human subject undergoing a routine medical check-up. Alternatively, the subject may be at risk of having a disease for example a genetically predisposed subject, a subject with medical and/or family history of cancer, a subject who has been exposed. According to another embodiment, the subject may be a patient diagnosed with the disease and is performing a routine check-up, in-between treatments.

In an aspect the invention relates to a kit comprising reagents for the extraction of nucleic acid from platelets and a panel of reagents that specifically detect and/or amplify one or more clonal haematopoiesis associated mutation, and optionally instructions for use.

The panel of reagents may specifically detect one or more of the following CH associated mutations are selected from JAK2^{V617F}, JAK2 exon12, CALR exon9, MPL^{S5050}, MPL^{W515}, CBL exon 8, CBL exon 9, KRAS exon 2, KRAS exon 3, GNB1 exon 5, DNMT3A all exons, TET2 all exons, ASXL1 exon 12, IDH2 exon 4, SF3B1 exon 14, SF3B1 exon 15, SRSF2^{P95}, U2AF1 exon 2, U2AF1 exon 6, PPM1D exon 6, TP53 all exons.

In an aspect the invention relates to a kit comprising reagents for the extraction of nucleic acid from platelets and a panel of reagents that specifically detect and/or amplify one or more cancer associated modification, or cancer specific mutation.

The panel of reagents may specifically detect one or more of the following cancer specific mutations; BRAFV600E, KRASG12D, PIKCAH1047R, TP53R273H.

The CH associated mutations or cancer associated mutations may be detected using a targeted gene sequencing panel, next generation sequencing, primers or probes designed to detect specific mutations.

In an aspect the invention relates to a method of treatment of a subject with cancer comprising the steps of:
providing a biological sample comprising thrombocytes,
extracting nucleic acid from said biological sample,
analysing said nucleic acid to identify the presence of one or more cancer associated nucleic acid fragments,
selecting a treatment; and
administering the treatment.

In an embodiment the methods for the detection or prognosis of cancer and CH described herein may be combined to provide a combined method for the detection of cancer and CH. This combined method may comprise any of the features herein described.

In an aspect the invention relates to a method of preparing a nucleic acid fraction comprising the steps of:
providing a biological sample comprising thrombocytes,
extracting nucleic acid from said biological sample to form a nucleic acid sample,
enriching said nucleic acid sample for one or more cancer associated nucleic acid fragments and/or clonal haematopoiesis associated mutations.

Various steps may be applied to the biological sample in order to isolate the thrombocytes from the sample and also to enhance the extraction of the nucleic acid from said thrombocytes. These steps may be applied to any of the methods described herein. In an embodiment the biological sample may be processed to provide a sample for analysis. For example, the biological sample may be purified, or digested, or specific compounds may be extracted therefrom. Depending upon the method of characterizing the nucleic present in the thrombocytes in said biological sample, the thrombocytes may be extracted from the sample by methods known to the skilled person and be transferred to any suitable medium for extraction of the nucleic acid. The biological sample may be treated to remove abundant nucleic acid degrading enzymes (like RNases, DNases) therefrom, in order to prevent early destruction of the nucleic acids.

In an embodiment a peripheral blood sample is collected in either EDTA or Streck or Heparin Lithium tubes. In embodiments, a peripheral blood sample is collected in either EDTA or Streck tubes. Centrifugation may be used to enable isolation of pure fractions of granulocytes, platelets and cell/platelet depleted plasma. In an embodiment the protocol to obtain platelets from a peripheral blood sample may be optimised to improve the purity of the platelets. Optimisation may be performed by altering centrifugation protocols (e.g. centrifugation speeds and brake settings) and altering the buffers used to isolate the platelets. For example, buffers which prevent platelet activation may be used in the method of the present invention.

The purity of the platelets for analysis may be greater than 90% pure, greater than 91% pure, greater than 92% pure, greater than 93% pure, greater than 94% pure, greater than 95% pure, greater than 96% pure, greater than 97% pure, greater than 98% pure, greater than 99% pure, greater than 99.5% pure, or greater than 99.9% pure, wherein purity is assessed in terms of the amount of platelets vs other blood cell types. Having a highly pure sample of platelets for analysis reduces contamination of the sample with other cell types. In an embodiment the protocol to obtain platelets from a peripheral blood sample may be optimised to improve the purity of platelets and/or reduce contamination with white blood cells and/or red blood cells.

In embodiments in which the method comprises centrifuging the biological sample, the method may further comprise isolating an upper percentage of the platelet-rich plasma following centrifugation. For example, the method may comprise isolating the upper 95%, 90%, 85%, 80%, 85%, 70% of the platelet-rich plasma following centrifugation. Such isolation steps may improve platelet purity and reduce contamination with other cell types.

In embodiments the method may involve using CD45 and/or CD42 beads to deplete white blood cells and enrich platelets.

In embodiments the method may involve using a leucocyte filter to reduce contamination from white blood cells.

In an embodiment the method for preparing a nucleic acid fraction comprises a step of enriching the nucleic acid sample for shorter nucleic acid fragments, in particular this step may be used when preparing a nucleic acid fraction enriched for one or more cancer associated nucleic. For example the method may comprise a step of enriching the nucleic acid sample for fragments with a length between 20 bp and 500 bp, 20 bp and 400 bp, 20 bp and 300 bp, 20 bp and 200 bp, 20 bp and 150 bp, 50 bp and 500 bp, 50 bp and 400 bp, 50 bp and 300 bp, 50 bp and 200 bp, 50 bp and 150 bp, 100 bp and 500 bp, 100 bp and 400 bp, 100 bp and 300 bp, 100 bp and 200 bp, or 100 bp and 150 bp. In a preferred embodiment the nucleic acid sample is enriched for fragment length between 50 bp and 250 bp, or 100 bp and 200 bp.

An aspect of the invention relates to a method of genetically typing a sample of thrombocytes comprising:
providing a biological sample comprising thrombocytes;
extracting RNA from said biological sample;
converting RNA to cDNA;
analysing said cDNA to identify the presence of one or more clonal haematopoiesis associated mutations, thereby genetically typing the sample.

An aspect of the invention relates to a method of genetically typing a sample of thrombocytes comprising:
providing a biological sample comprising thrombocytes;
extracting nucleic acid from said biological sample;
analysing said nucleic acid to identify the presence of one or more cancer associated nucleic acid fragments, thereby genetically typing the sample.

The term “genetically typing” also referred to as genotyping refers to detecting differences in the nucleic acid present within a cell i.e., a thrombocyte, compared to a control. The control may be a healthy non diseased thrombocyte. The differences in the nucleic acid may be the present or absence of mutations, the upregulation or downregulation of certain nucleic acids and or the presence or absence of certain nucleic acid fragments.

The genetic typing may comprise analysing said nucleic acid to identify the presence of one or more cancer associated nucleic acid fragment, wherein the cancer associated nucleic acid fragment is a cell free nucleic acid fragment that has been released by a solid tumour and taken up by a thrombocyte. In an embodiment the thrombocytes are genotyped by the presence of one or more cancer associated nucleic fragments which may comprise one or more of the following mutations BRAFV600E, KRASG12D, PIKCAH1047R, TP53R273H.

In an embodiment the thrombocytes are genotyped by the presence of one or more CH associated mutations present in one or more of the following genes; JAK2 (Ensembl ID: ENSG00000096968), CALR (Ensembl ID: ENSG00000179218), MPL (Ensembl ID: ENSG00000117400), CBL (Ensembl ID: ENSG00000110395), KRAS (Ensembl ID: ENSG00000133703), GNB1 (Ensembl ID: ENSG00000078369), DNMT3A (Ensembl ID: ENSG00000119772), TET2 (Ensembl ID: ENSG00000168769), ASXL1 (Ensembl ID: ENSG00000171456), IDH2 (Ensembl ID: ENSG00000182054), SF3B1 (Ensembl ID: ENSG00000115524), SRSF2 (Ensembl ID: ENSG00000161547), U2AF1 (Ensembl ID: ENSG00000160201), PPM1D (Ensembl ID: ENSG00000170836), TP53 (Ensembl ID: ENSG00000141510). In an embodiment the thrombocytes are genotyped by the presence of one or more of the following mutations JAK2^{V617F}, JAK2 exon12, CALR exon9, MPL^{S5050}, MPL^{W515}, CBL exon 8, CBL exon 9, KRAS exon 2, KRAS exon 3, GNB1 exon 5, DNMT3A all exons, TET2 all exons, ASXL1 exon 12, IDH2 exon 4, SF3B1 exon 14, SF3B1 exon 15, SRSF2^{P95}, U2AF1 exon 2, U2AF1 exon 6, PPM1D exon 6, TP53 all exons.

The present inventors have also determined that analysis of maternal platelet nucleic acid can be used to identify genetic information related to their offspring. As such the platelet analysis may find utility in antenatal screening.

An aspect of the invention relates to a method for antenatal screening for foetal genetic information, comprising
providing a biological sample comprising thrombocytes, obtained from a pregnant woman; extracting nucleic acid from said biological sample;
analysing said nucleic acid for genetic information related to the foetus.

The method of the present invention involves extracting nucleic acid from thrombocytes obtained from pregnant woman or a woman who has recently given birth. Nucleic acid is then extracted from said thrombocytes. The nucleic acid is subsequently analysed for genetic information related to the foetus of the pregnant woman or the child of the woman that has recently given birth. The nucleic acid that is extracted may be RNA and/or DNA. Where RNA is extracted from thrombocytes, the RNA which is subsequently converted to cDNA for analysis. The DNA that is extracted from the thrombocytes may be genomic DNA (gDNA). Where a combination of DNA and RNA are extracted from the thrombocytes the method may comprise parallel analysis of cDNA and gDNA. The analysis of cDNA and gDNA may be performed simultaneously, sequentially or separately to detect genetic information related to the foetus of the pregnant woman or the child of the woman that has recently given birth. In one embodiment RNA and gDNA are extracted from thrombocytes and analysed in parallel for the presence of genetic information related to the foetus of the pregnant woman or the child of the woman that has recently given birth. In one embodiment RNA and gDNA are extracted from thrombocytes and analysed separately for the presence of genetic information related to the foetus of the pregnant woman or the child of the woman that has recently given birth. In one embodiment RNA is extracted from thrombocytes and analysed for the presence of genetic information related to the foetus of the pregnant woman or the child of the woman that has recently given birth. In one embodiment gDNA is extracted from thrombocytes and analysed for the presence genetic information related to the foetus of the pregnant woman or the child of the woman that has recently given birth.

The genetic information may be related to the sex of the foetus or child, genetic conditions such as Down's syndrome, Edwards' syndrome, Patau's syndrome, cystic fibrosis, spina bifida, sickle cell, thalasaemia.

The genetic information related to the foetus or child may be identified by analysing said nucleic acid for certain markers or genes, for example when looking to identify the sex of the foetus or child fragments of the Y-chromosome can be screened for in particular fragments of the SRY gene.

The biological sample may be obtained from said pregnant woman at a certain point through the pregnancy or may be obtained shortly after birth. The biological sample may be obtained at 1, 2, 3, 4, 5, 6, 7, 8, 9, 10, 11, 12, 13, 14, 15, 16, 17, 18, 19, 20, 21, 22, 23, 24, 25,26, 27, 28, 29, 30, 31, 32, 33, 34, 35, 36, 37, 38, 39, 40 weeks gestation. The biological same may be obtained 1, 2, 3, 4, 5, 6, 7, 8, 9, 10, 11, 12, 13, 14 days postpartum.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The term “comprising” or “comprises” where used herein means including the component(s) specified but not to the exclusion of the presence of other components. The term “consisting essentially of” or “consists essentially of” means including the components specified but excluding other components except for materials present as impurities, unavoidable materials present as a result of processes used to provide the components and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

The invention is further illustrated in the following non-limiting examples.

### Examples

### Materials and Methods

Blood collection

Samples were collected by the BRC Oxford GI Biobank and Oxford Radcliffe Biobank (ORB). All procedures were approved by the Yorkshire & The Humber-Sheffield Research Ethics Committee (REC reference number: 16/YH/0247), ORB research tissue bank ethics, (REC reference 19/SC/0173) and INForMeD Study (REC: 16/LO/1376 (199833)). Participants signed informed consent whenever required. 10-20 ml of peripheral blood was drawn into EDTA coated (Fisher Scientific, cat #367839) or Cell-Free DNA (Streck, cat #218997) blood collection tubes from solid tumour patients presenting with colorectal, oesophageal or pancreatic cancer at their pre-op assessment. 10 ml of peripheral blood was also collected from serrated polyposis syndrome patients. For healthy controls, fully anonymised blood samples were collected from the Oxford Biomedical Research Laboratory (Clinical Diagnostic Lab). Samples were collected from individuals in either inpatient or outpatient care and were excess to clinical requirements. No clinical information was recorded in accordance with HTA requirements. Murine blood was drawn from KPN (villinCre^{ER} Kras^{G12D/+} Trp53^{fl/fl} R26^{N1icd/+}) and KP (villinCre^{ER} Kras^{G12D/+} Trp53^{fl/fl}) mice via cardiac puncture into EDTA coated microvettes (Sarstedt, cat #20.1288).

All procedures were performed in accordance with the ethical standards of the Declaration of Helsinki. Patient samples were collected by the Oxford Translational Gastroenterology Unit (TGU) and Oxford Radcliffe Biobank (ORB). All patients provided informed consent and procedures were approved by Yorkshire & The Humber-Sheffield Research Ethics Committee (REC reference number: 16/YH/0247) and ORB research tissue bank ethics (REC reference 19/SC/0173). 10 to 20 ml of peripheral blood was drawn into EDTA-coated (Fisher Scientific, cat #367839) or Cell-Free DNA (Streck, cat #218997) blood collection tubes from patients presenting with colorectal, oesophageal or pancreatic cancer. 10 ml of peripheral blood was collected from patients with sessile serrated lesions and from those on the bowel cancer screening pathway or those with inflammatory bowel disease undergoing colonoscopy. For healthy controls, fully anonymised blood samples were collected from the Oxford Biomedical Research Laboratory under The INForMeD Study (REC reference 16/LO/1376). The usage and collection of healthy human donor blood samples in Edinburgh was authorised under the project “21-EMREC-041—The Role of Inflammation in Human Immunity”.

For obstetric samples, 10 ml of blood was collected from mothers at the time of caesarean delivery, and postnatally at 24 and 48 hours after delivery into Streck blood collection tubes. Samples were taken at the Department of Women's and Reproductive Health at the John Radcliffe Hospital Oxford and approved by the Oxfordshire Research Ethics Committee (07/H0607/74). Sex of the neonate was confirmed after delivery and all mothers provided written informed consent.

### Animals

All mice were bred and maintained in accordance to UK Home Office regulations. All experiments were performed under Project Licenses P2FF90EE8 and POB63BC4D and approved by the University of Oxford Animal Welfare and Ethical Review Body. All mice were housed in individually ventilated cages at the animal unit either at Functional Genetics Facility (Wellcome Centre for Human Genetics, University of Oxford) or the Biomedical Services Facility at the John Radcliffe Hospital (The MRC Weatherall Institute of Molecular Medicine, University of Oxford), in a specific-pathogen-free (SPF) facility, with unrestricted access to food and water, and were not involved in any previous procedures.

For the immune thrombocytopenia model, CD45.2 female, wild-type mice were injected intravenously with either an anti-platelet antibody to reduce platelet count (Emfret, cat #R300) or an IgG control that had no cytotoxic effects on platelets in mice (Emfret, cat #C301). Mice were humanely culled and then blood was collected via cardiac puncture into EDTA coated microvettes (Sarstedt, cat #20.1288) at 24, 72 and 120 hours post treatment. Platelet counts were determined using an automated blood cell counter. cfDNA was extracted from plasma and quantified. For the colorectal cancer (CRC) model, murine blood was drawn from KPN (villinCre^{ER} Kras^{G12D/+} Trp53^{fl/fl} R26N1^{icd/+}) and KP (villinCre^{ER} Kras^{G12D/+}Trp53^{fl/fl}) mice via cardiac puncture into EDTA coated microvettes (Sarstedt, cat #20.1288). All strains used in this model were maintained on C57BL/6J background. Both male and female KP and KPN mice were used.

Human Platelet and cfDNA Isolation

Human platelets were isolated within 4 or 24 hours of blood sampling (depending on blood tube). Whole blood was supplemented with citrate-dextrose solution (ACD) (Sigma, cat #C3821-50ML) and centrifuged at 180-200×g for 10-20 minutes at room temperature. 80% of the supernatant was removed and diluted with platelet wash buffer and centrifuged at 600-1200×g for 10-20 minutes at room temperature. The resulting platelet pellet was resuspended in pre-warmed HEPES-modified Tyrode's buffer. The remaining platelet poor plasma was centrifuged at 16000×g for 10 minutes at 4° C. to ensure the pelleting of all cellular components and the isolation of pure cfDNA. Platelet number in both samples was determined using an automated blood cell counter (Horiba, Pentra ES 60 Cell Counter).

### Mouse Platelet Isolation

Mouse platelets were also isolated within 4 hours of collection. Whole blood was supplemented with ACD and centrifuged at 100×g for 10 minutes at room temperature. The plasma was removed and collected into a clean Eppendorf. The plasma was then diluted with platelet wash buffer and centrifuged at 3500×g for 11 minutes to pellet the platelets. The platelet pellet was resuspended in pre-warmed HEPES-modified Tyrode's buffer. The platelet poor plasma was removed and centrifuged at either 1300×g or 16000×g for 10 minutes at 4° C. depending on the experiment to remove all cells and the final supernatant collected for cfDNA isolation. Platelet number was determined using an automated blood cell counter.

Blood tubes were kept at room temperature and handled gently to avoid platelet activation. For human samples, platelets were isolated within 4 or 24 hours of blood sampling (depending on blood tube) using a modified previously published protocol. Briefly, whole blood was supplemented with citrate-dextrose solution (ACD) (Sigma Aldrich, cat #C3821-50ML) and centrifuged at 180×g for 20 minutes at room temperature. Eighty % of the supernatant was removed, diluted with platelet wash buffer and centrifuged at 600×g for 20 minutes at room temperature. The resulting platelet pellet was resuspended in pre-warmed HEPES-modified Tyrode's buffer. The remaining platelet poor plasma was centrifuged at 16′000×g for 10 minutes at 4° C. to ensure the pelleting of all cellular components and the isolation of pure cfDNA. Platelet number in both samples was determined using an automated blood cell counter (Horiba, Pentra ES 60 Cell Counter).

### Human Granulocyte Cell Isolation

Human granulocytes were isolated from freshly drawn venous blood into EDTA coated tubes. Whole blood was supplemented with ACD and centrifuged at 180×g for 20 minutes at room temperature, low brake. The supernatant was removed and the remaining layer diluted with wash buffer. Diluted blood was layered onto density gradient media (GE Healthcare, cat #5442-03) and centrifuged for 20 minutes at 600×g, low brake. The supernatant was removed and the remaining RBC, granulocyte pellet was resuspended with chilled RBC Lysis Buffer and incubated at 4° C. for 5 min. The lysed sample was centrifuged at 1000×g for 5 min, the supernatant discarded and the granulocyte pellet diluted with wash buffer. If necessary, the lysis step was repeated with additional RBC Lysis Buffer to ensure the efficient removal of contaminating RBCs. The remaining white granulocyte pellet was washed, centrifuged and stored at −80° C. until required.

### Platelet Purity Assessment

Leukocyte contamination in the platelet pellet was assessed via flow cytometry using the LSR Fortessa X20. Single colour-stained controls and fluorescence minus one (FMO) controls were used for all experiments. Washed human platelets were stained with antibodies for 20 minutes at room temperature in the dark, prior to being washed and resuspended in FACS buffer (IMDM no phenol+10% FCS) for analysis. Gates were set using FMO-controls and negative populations. Viability was assessed using DAPI. Analysis was performed using FACSDiva v8.1 (BD Biosciences) and FlowJo v10.7.1 Software. Two gating strategies were employed to ensure platelets and contaminating leukocytes were accurately counted. Contamination of nuclear cells in the platelet pellet was found to be 0.003%. RNA was extracted from the platelet pellet and gene expression measured by RT-PCR to confirm expression of platelet-specific genes.

Purity of human platelet pellets were assessed via flow cytometry using an LSR Fortessa X20 flow cytometer (Becton, Dickinson and Company). Single colour-stained controls and fluorescence minus one (FMO) controls were used for all experiments. Washed human platelets were stained with antibodies for 20 minutes at room temperature in the dark, prior to being washed and resuspended in FACS buffer (IMDM no phenol+10% FCS) for analysis. Gates were set using FMO-controls and negative populations

DNA Extraction from Platelets, Cell Free Plasma and Nucleated Cells

DNA was extracted from equal volumes of fresh platelets (resuspended in HEPES-modified Tyrode's buffer) and ‘platelet poor plasma’ using the QIAamp Circulating Nucleic Acid Kit (Qiagen, cat #55114), as per manufacturer's instructions. Genomic DNA was extracted from nucleated cells using the DNeasy Blood & Tissue Kit (Qiagen, cat #51104). Extracted DNA was quantified using a Qubit fluorometer (Thermofisher Scientific) and stored at −80° C. until required.

PA-DNA, cfDNA and gDNA Extraction and Quantification

DNA was extracted from equal volumes of fresh platelets and ‘platelet poor plasma’ using the QIAamp Circulating Nucleic Acid Kit (Qiagen), according to the manufacturer's instructions. Genomic DNA was extracted from granulocytes using the DNeasy Blood & Tissue Kit (Qiagen). Extracted DNA was quantified using the Qubit fluorometer (Thermofisher Scientific) and stored at −80° C. until required.

cfDNA and PA-DNA Fragmentation Analysis

The fragmentation profiles of PA-DNA and paired cfDNA from solid tumour patients were analysed using the Agilent Tapestation 2200 system (Agilent Technologies). cfDNA, HS D5000 and genomic DNA reagents and screen tape were used for analysis (Agilent, cat #5067-5630, 5067-5592 and 5067-5365). Electrophoresis data was analysed using the Tapestation Analysis Software.

### Cell Lines

The following cancer cell lines were used as positive and negative controls for common cancer-associated mutations; HCT116 (CVCL S744) PIK3CAH1047R, LS180 (CVCL 0397) KRASG12D, COLO 205 (CVCL 0218) BRAFV600E, HEL (CVCL_0001) JAK2V617F. All cell lines were purchased from American Type Culture Collection (cat #CCL-247, CL-187, CCL-222 and TIB-180 for HCT116, LS180, COLO 205 and HEL cells respectively). All cells were cultured according to American Type Culture Collection recommendations and tested monthly for mycoplasma contamination.

The human Burkitt's lymphoma cell line, BL2 (CVCL 1966, cat #CRL-2959) was used to generate tumour cell-derived extracellular vesicles (EVs). Both standard BL2 cells and BL2 cells stably transfected with the apoptosis-suppressing gene bcl-2 were used, to obtain apoptotic (apo-EV) from BL2 cells and non-apoptotic EVs from apoptosis-resistant BL2-Bcl-2 cells. Protein expression was regularly tested by flow cytometry, confirming that 98% of the BL2-bcl-2 cells expressed Bcl-2. Both cell lines were maintained in 50% X-VIVO medium (50% Gibco RPMI-1640, 50% X-VIVO-20 medium; Lonza, Basel, Switzerland), supplemented with 50 U/ml penicillin and 50 μg/ml streptomycin at 37° C. with 5% CO2.

### In Vitro Co-Culture

All blood cell and tumour cell co-culture experiments were conducted using 3×10⁵ platelets/ul, 0.075×10⁵/μl MNCs, 3×10⁵ RBCs/μL and 0.03×10⁵/μl tumour cells. All cells were re646 suspended in Tyrode's salts (Sigma Aldrich, cat #T2397-100ML).

### Blood Cell-Tumour Cell Incubation

Platelets, MNCs and RBCs from healthy donors were added to individual wells within a 24-well plate and a 1.0 μM TC Insert (Sartstedt, cat #83.3932.101) placed into the well. Cancer cells were carefully placed in the inserts and the plate incubated for approximately 8 hours at 37° C. Control samples were incubated simultaneously but without the addition of tumour cells. After incubation, the inserts were removed, and the blood cells transferred to Eppendorfs. To ensure complete removal of any contaminating cfDNA in the media, all cells/platelets were washed and centrifuged at 600×g for 10 min. The supernatant was removed, and the pelleted blood cells re-suspended in fresh buffer. The cells were washed for a total of three times and finally resuspended in 1 ml of fresh pre-warmed HEPES-modified Tyrode's buffer prior to DNA extraction using the QIAamp Circulating Nucleic Acid Kit (Qiagen, cat #55114).

### DNase Treatment of Platelets and Conditioned Media

Following incubation with HEL erythroleukaemia cells, platelets were treated with DNase according to the manufacturers protocol (Ambion DNA-free Kit, cat #AM1906). To confirm catalytic activity of the enzyme, DNase was also added to conditioned media. Following DNase treatment, the platelet samples were washed with 500 μl of 5 mM EDTA and centrifuged at 600×g for 10 minutes. The supernatant was removed and the wash repeated. After the second centrifugation, the supernatant was discarded and the platelet pellet resuspended in 1 ml of fresh pre-warmed HEPES-modified Tyrode's buffer. Following DNase inactivation of the conditioned media, 900 μl of HEPES buffer was added to a final volume of 1 ml in preparation for DNA extraction. For control samples, no DNase was added and 10 μl of 1M tris-HCL was added in replacement of 10X DNase I Buffer for the platelet samples.

Generation of Extracellular Vesicles (EVs) from BL2 and BL2Bcl2 Cells

BL2 and BL2-Bcl-2 cells were cultured at 20×10⁶ 673/ml in 0.1 μm filtered 50% X-vivo 20 and irradiated with ultraviolet light to induce apoptosis with 6 doses at 50 mJ/cm2, for a total of 300 mJ/cm2. Apoptosis was monitored hourly by Annexin V and Sytox Blue staining. To isolate EVs, the culture supernatant was centrifuged at 25×g for 1 hour followed by sequential filtering through a 5 μm mesh filter and a 1.2 μm syringe filter. EV concentration was measured 678 by Nanoparticle Tracking Analysis on a Nanosight LM14.

### Purification and Labelling of EVs

The EVs were purified from soluble factors and proteins from the cell culture by size exclusion chromatography using in-house prepared sepharose columns (Thermofisher, cat #45-000-067 and Sigma Aldrich, cat #CL6B200-100ML). The purified EVs suspended in 0.1 μm filtered HBSS were stained with Biotium CF568 NHS ester with a covalent attachment of the dye on the EV proteins, according to the manufacturer's protocol (Biotium, cat #NC1542764). To remove unbound dye, the EVs were purified again by size exclusion chromatography through sepharose columns.

### EV-Platelet Incubation and DNA Extraction

50×10⁸ labelled, purified EVs resuspended in in Tyrode's buffer were incubated with 0.1×10⁸ platelets for three hours at 37° C. with gentle inversion every 30 minutes. Following incubation, the samples were centrifuged for 20 minutes at 800×g. The supernatant was discarded, and the platelets re-suspended in 300 μl of phosphate buffered saline (PBS) medium (Life Technologies, cat #10010-056). EV and platelet pellets were treated with DNase before DNA extraction as per manufacturer's protocol (Sigma Aldrich, cat #AMPD1). DNA extraction was 20 performed using a QiaAMP DNA blood kit as per manufacturer's protocol (Qiagen, cat #51104), samples were eluted in 30 μl of elution buffer, and the DNA concentration was measured using a Qubit Fluorometer.

### Cell Culture

### Platelet-Tumour Cell Incubation

To confirm that specific cancer-associated gene rearrangements were detectable within platelets, platelets were incubated with a variety of colorectal cancer cell lines. Platelets from healthy donors were added to individual wells within a 24-well plate and a 1.0 μM TC Insert (Sartstedt, cat #83.3932.101) placed into the same well. Tumour cells were carefully placed inside the inserts and the plate left to incubate for approximately 8 hours at 37° C. Note that control samples were incubated without the addition of tumour cells. After incubation, the inserts were removed and the platelets transferred to Eppendorfs. To maximise the collection of the platelets, the wells were washed with pre-warmed HEPES-modified Tyrode's buffer. To ensure the removal of potential contaminating cfDNA in the media, all platelets were washed and centrifuged. The supernatant was removed and the pelleted platelets re-suspended in fresh buffer. The platelets were washed for a total of 3 times and finally resuspended in 1 ml of fresh pre-warmed HEPES-modified Tyrode's buffer in preparation for DNA extraction using the QIAamp Circulating Nucleic Acid Kit (Qiagen).

### DNase Treatment of Platelets and Conditioned Media

To determine whether platelets internalise cfDNA, platelets incubated with tumour cells were treated with DNase (Ambion DNA-free Kit, cat #AM1906). To confirm catalytic activity of the enzyme, DNase was also added to conditioned media as described in the manufactures protocol. After treatment the platelet samples were then washed with 500 μl of 5 mM EDTA and pelleted. The supernatant was removed and the wash repeated. After the second centrifugation, the supernatant was discarded and the platelet pellet resuspended in 1 ml of fresh pre-warmed HEPES-modified Tyrode's buffer. For control samples, no DNase was added and 10 μl of 1 M tris-HCL was added in replacement of 10X DNase I Buffer for the platelet samples.

### Digital Droplet (Dd) PCR

Human ddPCR

To explore the possibility of platelets acquiring specific mutations via the uptake of DNA fragments, ddPCR assays were ordered from Bio-Rad. All ddPCR assays were conducted on a QX200 Droplet Digital PCR System using the manufacturer's recommended protocol and reagents. Positive and negative controls were included in every assay. Analysis was performed using the QuantaSoft software (Bio-Rad Laboratories, Watford UK).

KRAS^{G12D} Mouse ddPCR

A ddPCR assay was designed for the detection of the KRAS p.G12D c.35G>A point mutation in PA-DNA and cfDNA isolated from KRAS^{G12D} mice. To detect mouse KRASG12D, a ddPCR assay was designed for the detection of the KRAS p.G12D c.35G>A point mutation. Primers for the detection of KRAS were as follows: forward 5′-GCCTGCTGAAAATGACTGAG-3′ (SEQ ID NO: 1) and reverse 5′-CGTAGGGTCATACTCATCCAC-3′ (SEQ ID NO: 2). Two dual labelled probes were also used to target the wild-type and mutant sequences. Wild-type 5′-HEX-ACGCC[+A][+C]CAG[+C]TCCAA-BHQ1-3′ (SEQ ID NO: 3) and mutant 5′-6FAM-AC[+G]CC[+A][+T][+C]AG[+C]TCCAA-BHQ1-3′ (SEQ ID NO: 4). Square brackets indicate LNA (locked nucleic acid) bases (Merck Life Sciences).

### Immunofluorescence Microscopy

### Platelet-Tumour Cell Interaction

Human platelets were isolated from healthy donors as previously described and stained with CD42b/AF488 (Biolegend, cat #303914) for 30 minutes at room temperature. Separately, COLO205 cells were labelled using a highly specific live cell DNA stain, SiR-DNA (Spirochrome, cat #CHF260.00). Stained COLO205 cells were then washed twice with MEM Alpha 1X (without phenol) (Gibco, cat #41061-029) supplemented with 5% FCS and added to microscopy slides (Greiner Bio-One, cat #543079). Stained platelets were added to the wells containing tumour cells immediately before imaging. To analyse endogenous wild-type DNA within healthy donor platelets, platelets were co-stained with anti-CD42/AF488 and NUCLEAR-ID® Red DNA and imaged alone.

Platelet Uptake of Tumour Derived cfDNA

Nuclear DNA of COLO205 cells was labelled using the NUCLEAR-ID® Red DNA stain. After incubation at 37° C. for 45 minutes, the cells were washed with MEM Alpha 5% FCS, pelleted and the supernatant removed. The cells were washed a further two times to ensure complete removal of the DNA stain and the COLO205 cells were resuspended in 200 μl of fresh MEM Alpha 5% FCS. The cells were incubated at 37° C. and vortexed periodically to encourage cell death and DNA release. After ˜2 hours, the COLO205 cells were centrifuged at 16000×g for minutes at 4° C. Supernatant containing cfDNA was removed from the pelleted cells, transferred to a fresh Eppendorf and the COLO205 cells discarded. Healthy donor platelets were stained with anti-CD42/AF488 as previously described and added to wells of Poly-L-lysine coated slides (Ibidi, cat #81201). DNA labelled COLO205 cell conditioned media was added to the platelets immediately before imaging. All cells were resolved by fluorescent microscopy using the Zeiss Spinning Disk Confocal with a 63 x oil immersion objective.

### Imaging of Apo-EVs

BL2 cell-derived apo-EVs were isolated, labelled with an amine-reactive fluorescent dye (Biotium, cat #92131) and frozen as previously described. Prior to imaging, the EVs were thawed and co-stained with DAPI for 30 minutes at room temperature. The EVs were washed twice, pelleted at 20′000×g for 30 min at 4° C. and resuspended in HBSS buffer (Thermofisher, cat #88284). Labelled EVs were then added to Poly-L-lysine coated chamber slides (Ibidi, cat #81201) and incubated overnight at 4° C. Following incubation, the supernatant was carefully removed and the EVs fixed with 4% formaldehyde at room temperature for 30 minutes. The fixative was then removed and the EVs gently washed three times with PBS. To confirm imaging of EVs and not auto-fluorescent debris, PBS was stained with DAPI, incubated overnight, and fixed onto slides alongside the EV preparations. The slides were mounted and the EVs and PBS controls resolved by fluorescent microscopy using a Zeiss LSM900 with a 63 x oil immersion objective.

### Platelet Apo-EV Incubation

Human platelets were isolated from healthy donors as previously described and resuspended in 1 ml of HBSS buffer. BL2 cell-derived apo-EVs labelled with an amine-reactive fluorescent dye (Biotium, cat #92131) were thawed and 5×10⁹ EVs were added to 10×10⁶ platelets and incubated for three hours at 37° C. To ensure adequate mixing, the cells were inverted gently every 30 minutes. Following incubation, the cells were centrifuged at 800×g for 20 minutes, low brake. The supernatant (containing the EVs) was removed and the platelets resuspended in 100 ml of PBS. Platelets were centrifuged onto a Poly-L-lysine coated chamber slide (Ibidi, cat #81201) at 600 x g for 20 minutes and fixed in 2% formaldehyde/PBS for 10 minutes at room temperature. The fixative was then removed and the platelets washed twice with PBS. The platelets were then labelled with stained with anti-CD42/AF488 (BioLegend, cat #303914) for 20 minutes at room temperature. After labelling, the slides were washed, mounted with ProLong Gold Antifade Mountant (Thermofisher, cat #P36930) and imaged using the Zeiss LSM900.

### Fluorescence In Situ Hybridization (FISH)

Platelets in suspension were centrifuged onto Poly-L-lysine coated chamber slides (Ibidi, cat #81201) at 600 x g for 20 minutes and fixed in 2% formaldehyde/PBS for 10 minutes at room temperature. The platelets were then permeabilised in 0.5% Triton X-100/PBS for 10 minutes at room temperature and washed twice in 0.05% Triton-X-100/PBS. The platelets were washed in 0.02% Tween20/PBS (PBST) and incubated with the primary Anti-β-Tubulin antibody (Sigma Aldrich, cat #T5201) diluted in blocking buffer for 30 minutes at 37° C. in a humid box. Following primary labelling, the platelets were washed with PBST and incubated with the secondary Donkey Anti-Mouse IgG H&L AF405 antibody (Abcam, cat #ab175658) diluted in blocking buffer for 30 minutes at 37° C. The labelled platelets were then washed with PBST, fixed and washed with PBS for 10 minutes at room temperature. FISH staining was carried out according to the manufacturer's protocol. Briefly, slides were incubated in 0.1N HCL, washed with 2×SSC and dehydrated in 70% ethanol for 3 minutes. Equal volumes of X and Y-chromosome probes (Metasystems, cat #D-0323-050-FI and D-0324-100—OR) were added to the slide and covered with a 22×22 mm2 coverslip. The slides were incubated at 85° C. for 5 minutes, sealed with rubber cement and incubated in a humidified chamber at 37° C. overnight. Following hybridization, the rubber seal was removed, and the slides washed in 0.1×SSC at 60° C. Slides were mounted and imaged using the Zeiss LSM900 with a 63 x oil immersion objective. Control cells (HEL cells and MNCs) were fixed onto slides, permeabilised and labelled with the X- and Y-chromosome paints as previously described. Following overnight hybridization, the cells were washed and counterstained with DAPI.

### Image Analysis Software

Images were analysed using ImageJ v2.1.0 (National Institute of Health, US-MD) and 3D reconstructions were generated in Imaris Viewer v9.7.0 (Oxford Instruments, Abingdon). CellProfiler v4.0.7 (Broad Institute, US-MA) was used to quantitatively analyse platelet-tumour cell interaction. In brief, cells were segmented using the ‘Identify Primary/secondary Object’ module. Thresholding strategies were employed to accurately mask cells for segmentation. Platelets were tracked using the ‘Track Objects’ module. Finally, AF647 intensity was measured using ‘Measure Object Intensity’ and data ‘Exported to Spreadsheet’. This image analysis pipeline, as well as others can be recreated in Cell Profiler tool (freely available from the Broad Institute at www.cellprofiler.org).

cfDNA, pDNA and gDNA Fragmentation Analysis

803 Fragmentation profiles of pDNA and paired cfDNA were analysed using an Agilent Tapestation 2200 system (Agilent Technologies). cfDNA, HS D5000 and genomic DNA reagents and screen tape were used for analysis (Agilent, cat #5067-5630, 5067-5592 and 5067-5365, respectively). Electrophoresis data was analysed using the Tapestation Analysis Software (Agilent).

### Whole Genome Sequencing (WGS)

Whole genome sequencing (WGS) was performed on paired pDNA and cfDNA isolated simultaneously from the same peripheral blood samples of six patients with a recent diagnosis of gastrointestinal adenocarcinoma. Libraries were prepared from samples that were confirmed using the Agilent Tapestation to have both short DNA fragments of approximately 160 bp in addition to larger peaks of ˜12,000 bp.

gDNA Fragmentation

15 μl of DNA diluted in TE buffer (1 mM Tris-HCl, pH 8.0, 0.1 mM EDTA) was added to a Covaris microtube-15 (Covaris, cat #520145) and briefly centrifuged at 3000×g for 1 minute. The microtube was loaded onto the Covaris ME220 Focused-ultrasonicator and the protocol run using the treatment conditions required for a 150 base pair (bp) target peak. Fragment size was determined using the Agilent Tapestation 2200 system (Agilent Technologies) and the sonication was repeated if DNA fragments were >150 bp.

### Library Preparation

DNA-Seq libraries were prepared using the NEBNext Ultra II DNA Sample Preparation Kit for Illumina (New England BioLabs, Ipswich, MA, USA) according to the manufacturer's protocol. pDNA was size selected into 2 groups: short fragments (s-pDNA; under 600 bp) and large fragments (I-pDNA; over 600 bp). The short fragments were further cleaned to remove <100 bp fragments and large platelet fragments were fragmented via sonication as previously described. Following end repair and adapter ligation, adapter-ligated DNA fragments were amplified in enrichment PCR to generate final libraries. Cleaned up libraries were then multiplexed, and 1.5 pM libraries analysed on an Illumina NextSeq 500 (300 cycle PE) at low-pass (0.1X) for all samples, and 10X for the cfDNA and s-pDNA, using four lanes in each sample with 4% PhiX to monitor sequencing performance. This amounted to 240 fastq files.

### Bioinformatic Analyses

Quality control of all fastq files was conducted by running FastQC v0.11.8⁶⁶. Reads were adaptor- and quality-trimmed using Trim Galore67 v0.6.5 and aligned to the GATK Genome Reference Consortium Human Build 38 (GRCh38) 68 using bwa-mem v0.7.1769. Reads were aligned to alternate contigs to represent common complex variation using bwa_postalt.js (https://github.com/lh3/bwa/blob/master/bwakit/README.md) 69. Resulting BAM files were merged, sorted and indexed with Samtools v1.13.0⁷⁰.

GATK toolkit v4.1.7.0 was used to mark optical and PCR duplicates, estimate library complexity, and calculate summary metrics on insert sizes. Mapping rate was evaluated with samtools flagstat and the number of reads mapped to each chromosome and the mitochondrial genome with idxstats. Repetitive elements in the ENCODE blacklist were excluded using bedtools intersect v2.30.0⁷³.

### Insert Size Distribution

For all uniquely mapped, non-duplicated and properly paired read pairs, the frequency of insert size values was counted across all autosomes and chrX using Rsamtools. The distributions were plotted using ggplot2⁷⁴.

### Read Depth Around Transcription Start Sites

Transcription start sites were defined by refTSS v3.3⁷⁵. We used the nearest refTSS entry to the 5′ coding sequence of all protein coding genes. Coverage per base in a 2 kb region around each TSS was extracted with Rsamtools, counting only uniquely mapped, non-duplicate, correctly paired reads with insert sizes between 150 and 170 bp (“mononucleosome”) or between 280 and 350 (“dinucleosome”). PBMC gene expression values were taken from Blueprint Epigenome experiment EGAX00001327129 (http://dcc.blueprint epigenome.eu/ #/experiments/ERX1123729). Briefly, genes with posterior mean estimates of transcripts per million >15 were defined as “highly expressed” in PBMC

### Dinucleotide Peak Difference Quantification

A chi-square test of independence was performed in R with a simulated p-value to examine the relation between DNA type (cfDNA and s-pDNA) and the proportion of dinucleotide fragments. We separated the fragments in (i) fragments below 250 bp representing mononucleotide fragments; and (ii) fragments above 250 bp representing dinucleotide fragments. The proportion of dinucleotide fragments was higher in s-pDNA than in cfDNA (p 871<0.0005 in all samples.

### Chromosome Coverage

Coverage was calculated using samtools bedcov, using a bed file of 10 kb non-overlapping windows for all main chromosomes but excluding reads with mapQ<1. Position with mappability (as defined by umap: https://bismap.hoffmanlab.org/) in the lowest 8 percentile were removed. The Coverage was then corrected for mappability and GC content bias using a linear regression model and visualized with karyoplotR77 878 (Gel B, Serra E, 2017).

### Gini Index

After filtering for reads that are first in pair, uniquely mapped, mapped in proper pair, primary alignments, and non-duplicates, (Samtools view parameters: −f 2−F 3972−q 1), we calculated the coverage with Samtools depth. Evenness of coverage was evaluated by calculating the Gini index78 on the short pDNA and cfDNA's coverage, which was previously binned into adjacent 10 Kbp bins

ichorCNA

Copy number alterations in the 10X and low-pass WGS were evaluated using ichorCNA v0.2.041 (github: https://github.com/broadinstitute/ichorCNA/releases/tag/v0.2.0).

### Statistical Analysis

Parametric tests such as a paired Student t test were performed when comparisons were made between the same groups and the data visually appeared normally distributed. Nonparametric tests, such as a Wilcoxon signed rank test were used when data was paired but not normally distributed. Whilst a Mann-Whitney U test, was used when comparisons were made between different groups to determine statistical significance.

### Experimental Summary—Detection of Clonal Haematopoiesis

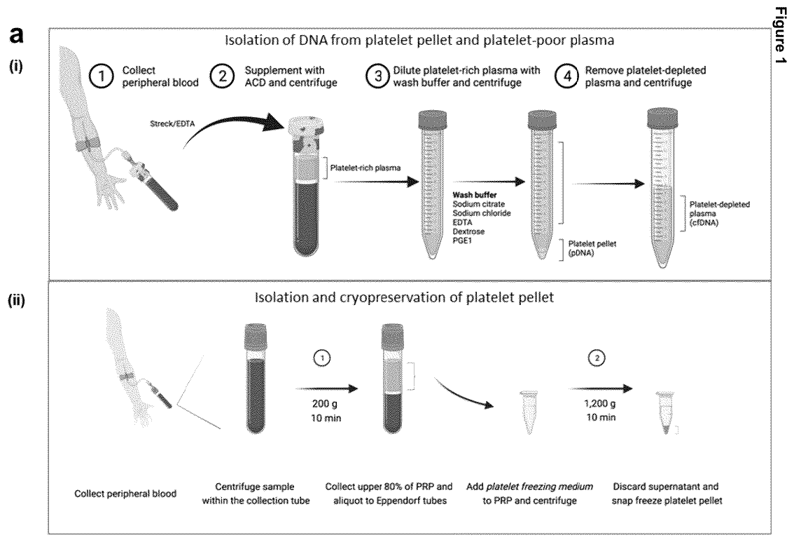

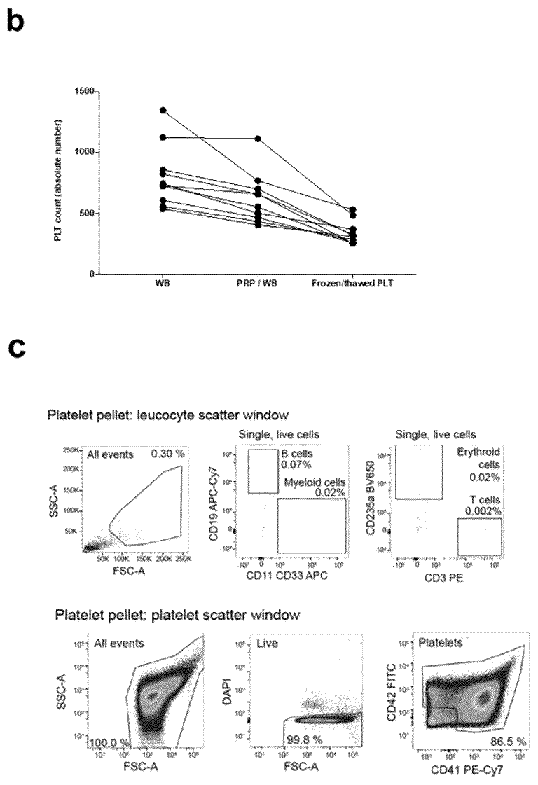

To study the utility of analysing platelet-associated nucleic acids in the detection of CH and solid tumours, the inventors first optimised a method for isolating purified platelets together with the currently used blood fraction for liquid biopsies in each disease setting—i.e. granulocytes for CH detection and cell-free DNA for solid tumours (FIG. 1A). It was confirmed that the isolated platelets show good recovery following cryopreservation and thawing (FIG. 1b), and that the platelet isolate has a very high purity (>99.96%) with minimal contamination with other blood cell types by flow cytometry, gene expression analysis and morphology of the cells (cytospin) (FIG. 1C-E). Further work is being done to further optimize a bespoke collection method for platelet-associated nucleic acids.

All CH studies to date are based on whole blood or granulocyte DNA and therefore they do not assess for platelet-restricted clones. As recent publications have indicated that a proportion of blood stem cells exclusively give rise to megakaryocytes and platelets, and that the proportion of these ‘megakaryocyte-primed’ stem cells increases with age, the inventors hypothesized that studying platelets in older individuals may increase the sensitivity of detection of CH. The teams have developed a method to compare the detection of CH in the cDNA of platelets with the nucleic acids (gDNA and cDNA) of granulocytes.

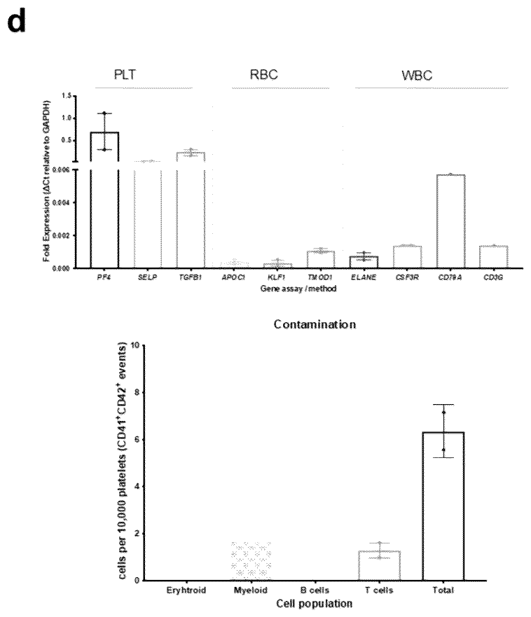

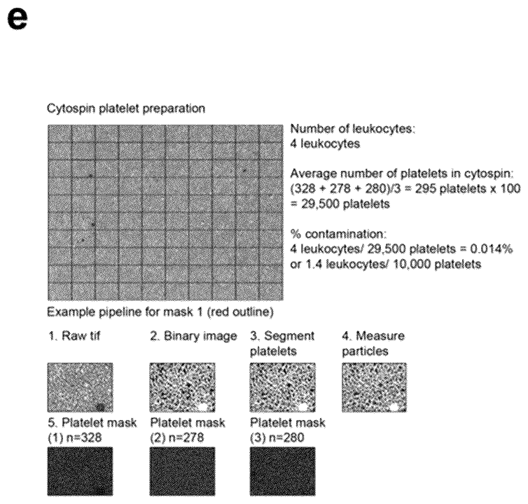

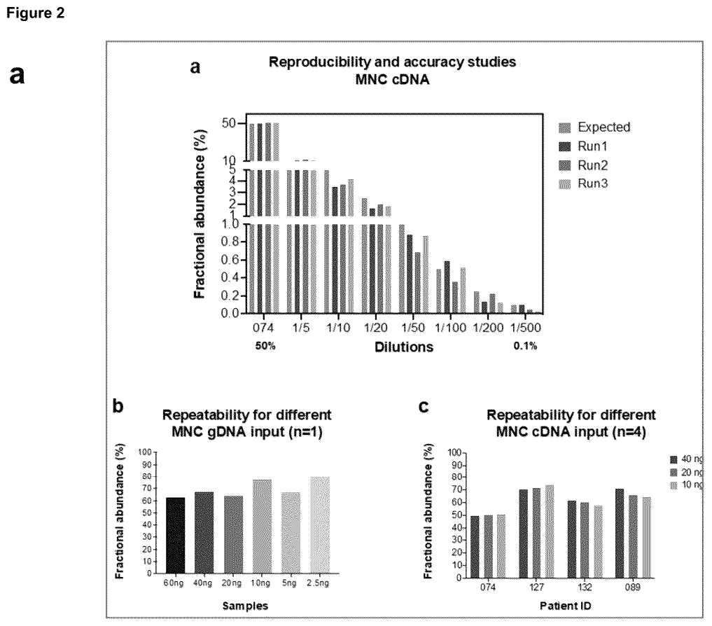

Digital droplet PCR (ddPCR) and a next generation sequencing panel were used to detect CH-associated mutations. ddPCR is—a highly sensitive and specific method for detection of genetic targets to study this. Both assays were optimised using experimental controls and demonstrated extremely high specificity (FIGS. 2a & 2b). Paired samples of platelet cDNA and granulocyte gDNA (i.e. isolated from the same sample) were analysed to compare the detection rates of JAK2V617F-driven CH in platelets vs. granulocytes. In 151 samples analysed, the fractional abundance of the JAK2V617F mutation was significantly higher in platelets than granulocytes (FIG. 2c). In 6/151 samples (3.9%), the mutation was only detected in the granulocytes, and in all cases, it was below the 2% cut-off that is typically used by diagnostic laboratories. In 13/151 samples (8.6%), it was detected in the platelets only, with several cases being above 5% fractional abundance. Only 2 samples had CH detected in both granulocytes and platelets (1.3%; FIG. 2c).

These findings confirm the presence of JAK2-V617F platelet-biased CH in humans, and almost 10% of the cases detected would have been missed by studying granulocytes alone. A key component of CH is the increased vascular disease, therefore platelet-biased CH may be particularly important in identifying patients who are at increased risk of cardiovascular events.

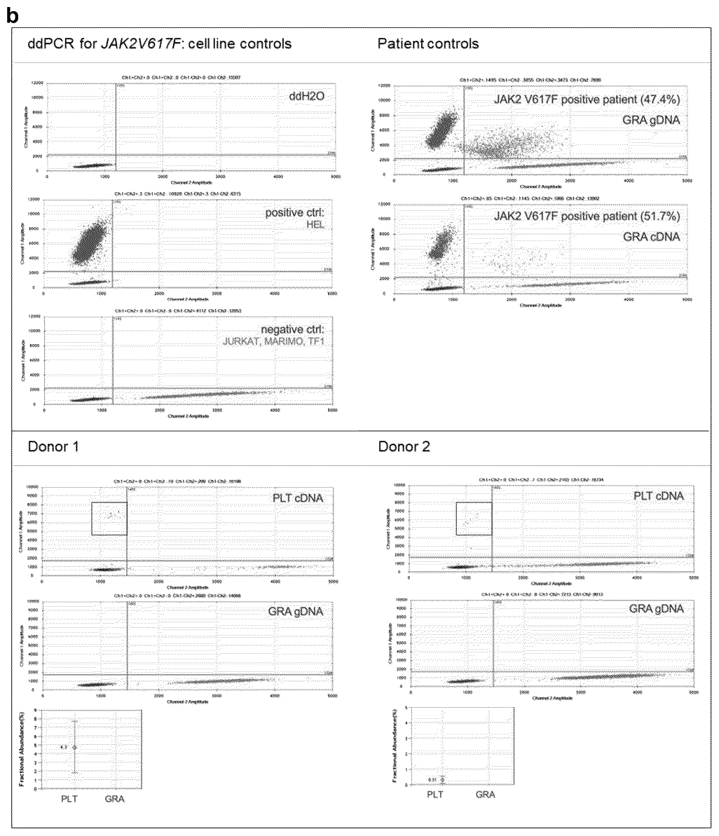

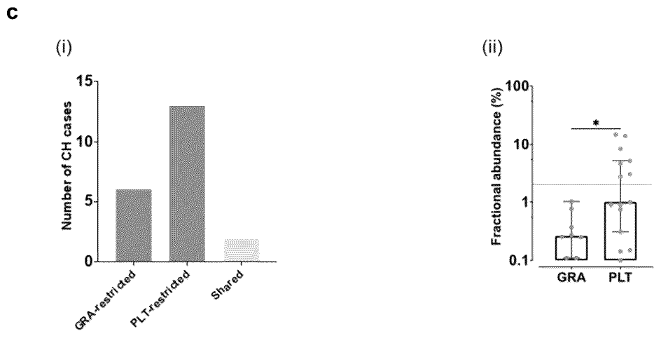

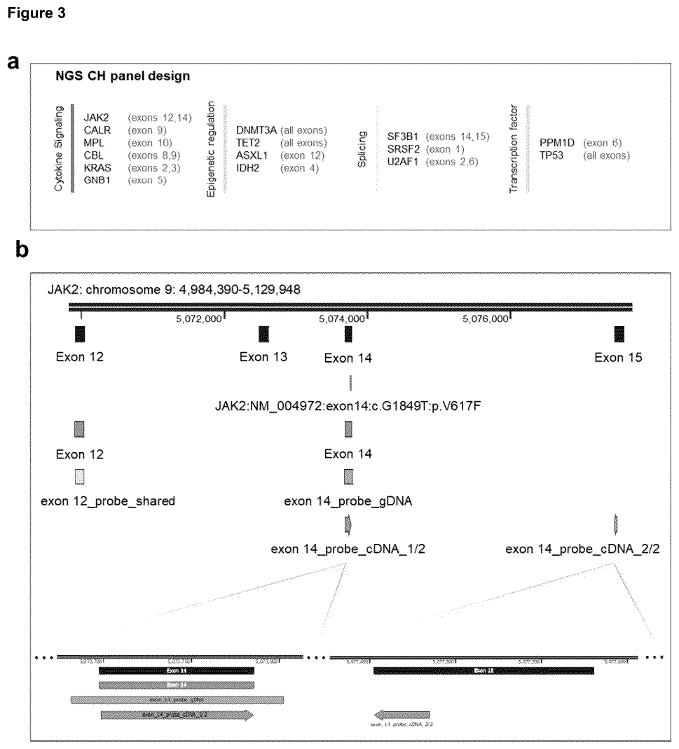

We also applied a bespoke hybridisation capture panel that enables parallel sequencing of both gDNA and cDNA (FIG. 3a). In 42 samples. we found CH in 15 (36%) of samples by analysing granulocytes, and in 67% of samples by analysing platelets, again indicating that many cases of CH will only be detected when platelet nucleic acids are analysed.

In addition to analysis of cDNA, CH and myeloid malignancy associated mutations can be detected by analysing platelet DNA, as shown in FIG. 4 for the detection of JAK2V617F mutations in patients with known myeloproliferative neoplasms.

### Experimental Summary—Detection of Non-Haematological Cancers

The second application is in early detection of solid tumours. Based on the literature suggesting that platelets bear many nucleic acid receptors and function as part of the innate immune system to ‘sense’ viral RNA, and a recent observation showing that non-human RNA derived from pollen (presumably acquired via the pulmonary circulation) was detectable inside platelets, the inventors hypothesized that platelets may also take up nucleic acid fragments released by tumour cells. This work focused on DNA rather than RNA as DNA is more stable, and as not all cancer-associated mutations are readily detectable at RNA level.

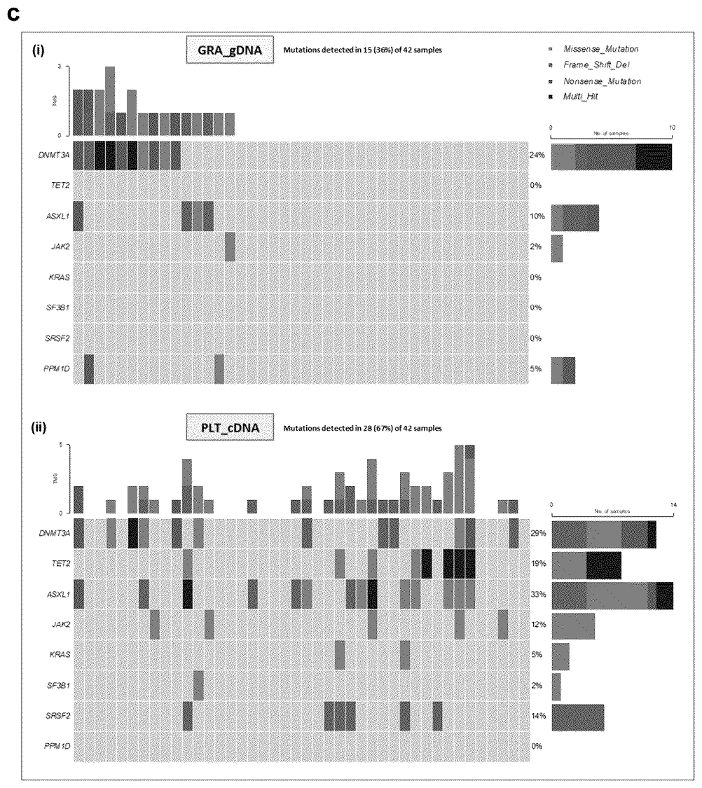

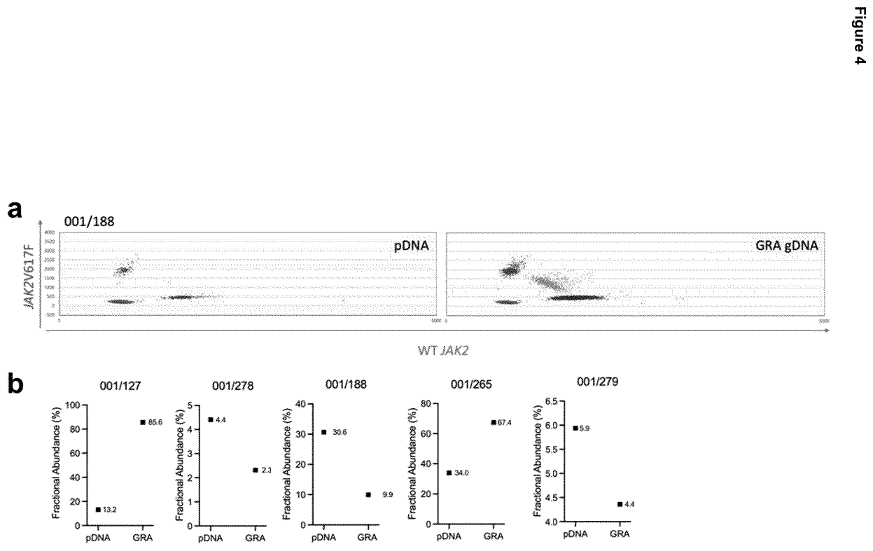

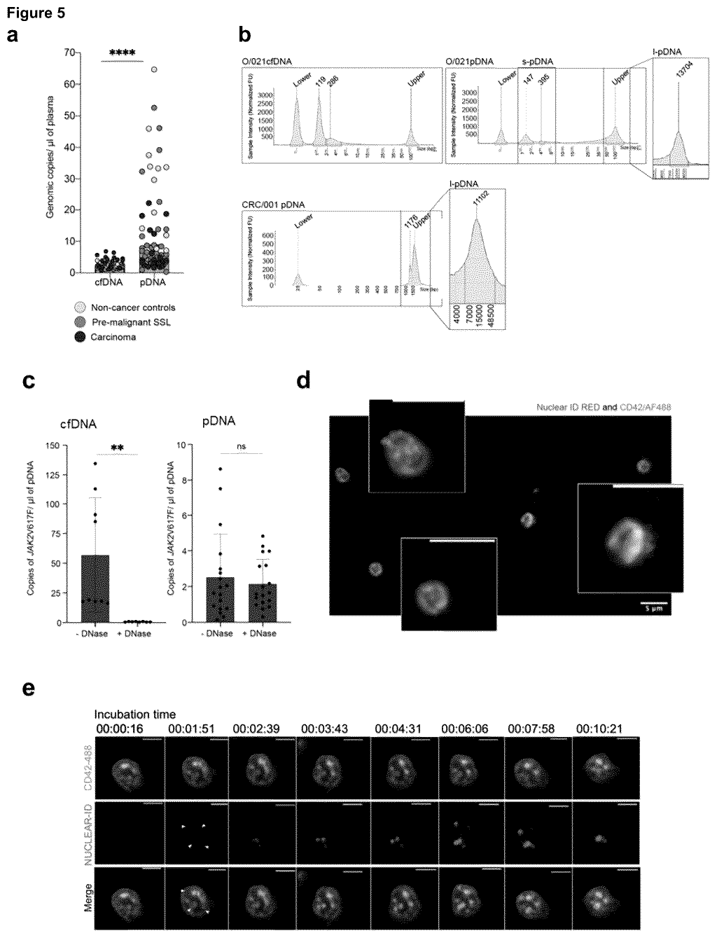

As platelets are anucleate cells and the association of platelets with DNA has not previously been reported, we first isolated DNA from a standard cell free DNA preparation and compared the quantity of DNA obtained to that isolated from the platelet pellet from the This demonstrated that DNA was substantially more abundant in platelets than free in the plasma (FIG. 5a). Further, in some donors, the fragmentation profile of platelet-associated DNA (pDNA) showed small fragments of around 150-160 bp, similar to those detected in cfDNA, with a nucelosomal footprint (FIG. 5b), while other donors showed DNA fragment lengths of ˜10,000-15,000 bp. Notably, the DNA detected in platelets persistent following treatment of the platelet pellet with DNAse, indicating that the DNA was likely contained within the platelet and protected from degradation (FIG. 5c). Live cell imaging of platelets co-incubated with a colorectal cancer cell line showed that DNA fragments were detectable (FIG. 5d), from 2 minutes and maximal within 10 mins of co-culture (FIG. 5e).

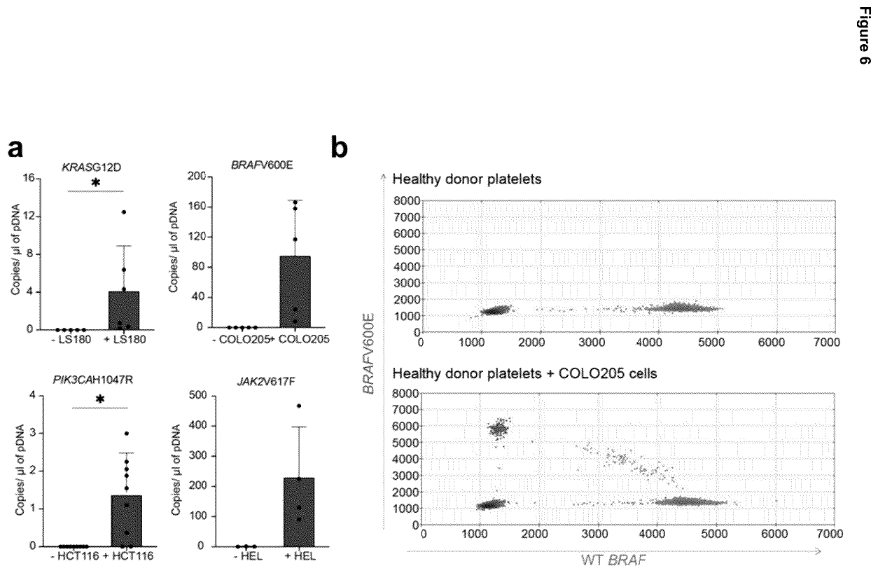

Next, we sought to confirm that specific cancer-associated gene rearrangements were detectable within platelets. Following in vitro co-culture of platelets derived from healthy donors with a variety of cancer cell lines (FIG. 4A), we confirmed that multiple cancer-specific gene mutations were detectable within p-DNA but not in platelets from healthy donors (FIG. 4B). This included BRAF, PIK3CA, TP53 and KRAS mutations from colon, ovarian as well as haematopoietic cancer cell lines (FIG. 6).

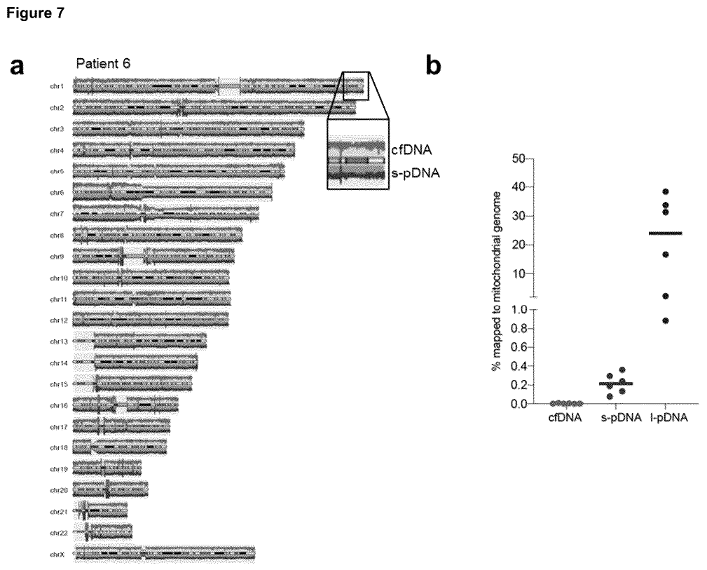

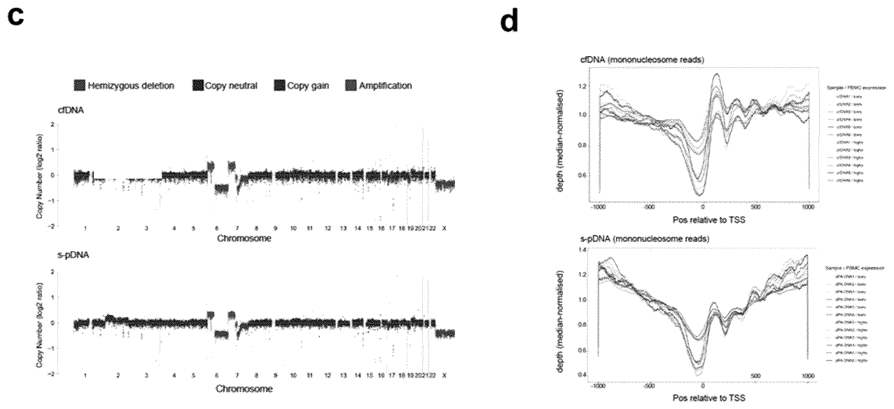

To further characterise platelet DNA, we performed whole genome sequencing and showed that pDNA contained fragments that mapped across the entire human nuclear genome (7a), as well as the mitochondrial genome (FIG. 7b). The majority of both the longer and shorter DNA fragments in pDNA mapped to the nuclear genome, although a higher proportion (˜40%) of the longer DNA fragments mapped to the mitochondrial genome than the fragments <200 bp (FIG. 7b). In a patient with active malignancy, copy number alterations present in the cell free DNA were also detectable in the pDNA (FIG. 7c), and fragmentation analysis showed depletion of reads around transcriptional start sites, confirming that analysis of DNA could give insights into the gene expression of the cell-of-origin of the DNA.

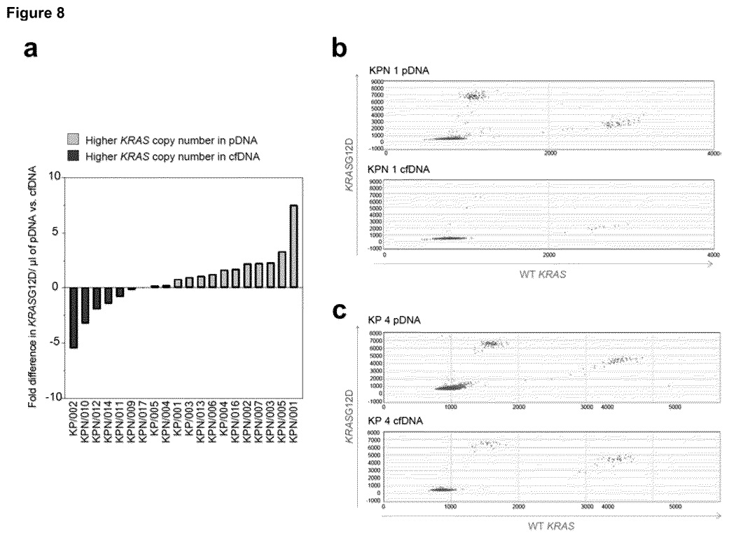

To explore the detection of cancer cell-derived DNA fragments in pDNA in vivo, we used a mouse model of colorectal carcinoma (mice express KRAS, TP53+/−NRAS mutations, FIG. 5A). Mutant KRAS G12D was readily detectable in mice with colorectal carcinoma (FIG. 5B), and in 13/20 mice the abundance of mutant KRAS copies was higher in platelets than in cfDNA, indicating that p-DNA may be more sensitive than cfDNA in some cases (FIG. 8).

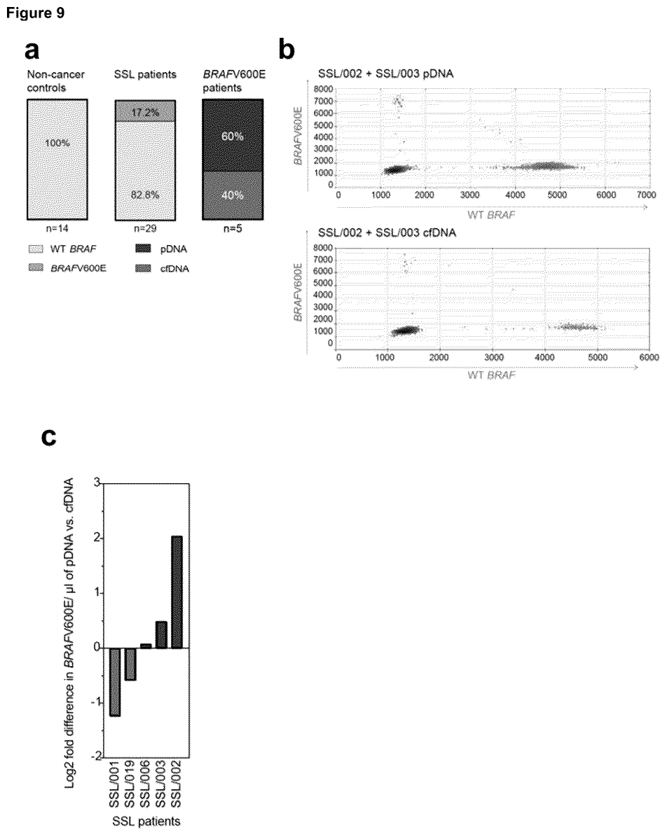

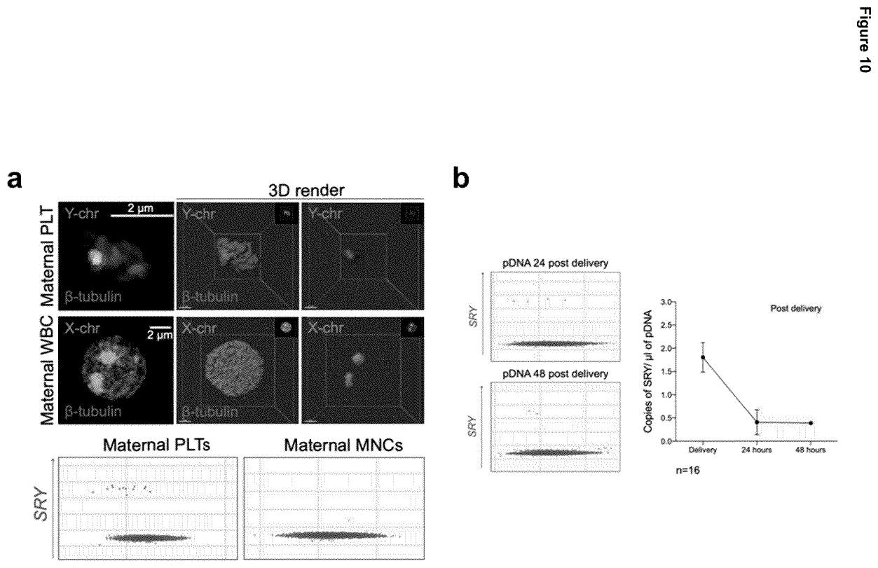

To address whether this occurs in setting of human cancer, samples were collected from patients with GI tumours as well as those with high-risk pre-malignant serrated polyps. It was confirmed that mutant BRAF was indeed detectable in patients with high-risk polyps in 17% of the patients studied so far (5/29), with an abundance of mutant BRAF alleles being higher in pDNA than cfDNA in 3/5 cases (FIG. 9A). Remarkably, this indicates that pDNA may be a highly sensitive test for early-stage malignancies and adds considerable value above that of standard cfDNA methods (FIG. 9).

### Detailed Examples

### Example 1—Platelets Contain DNA

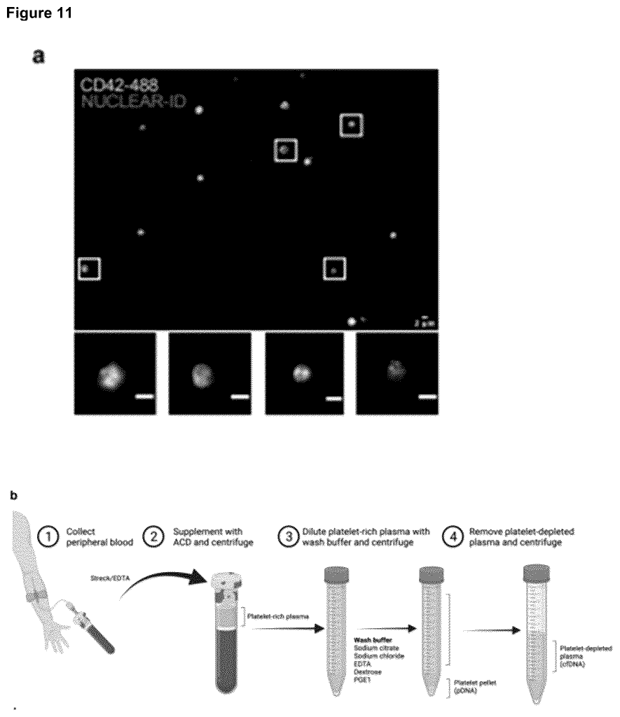

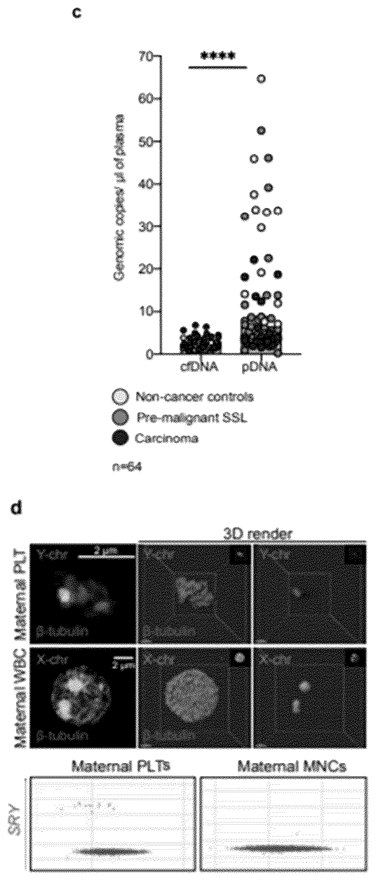

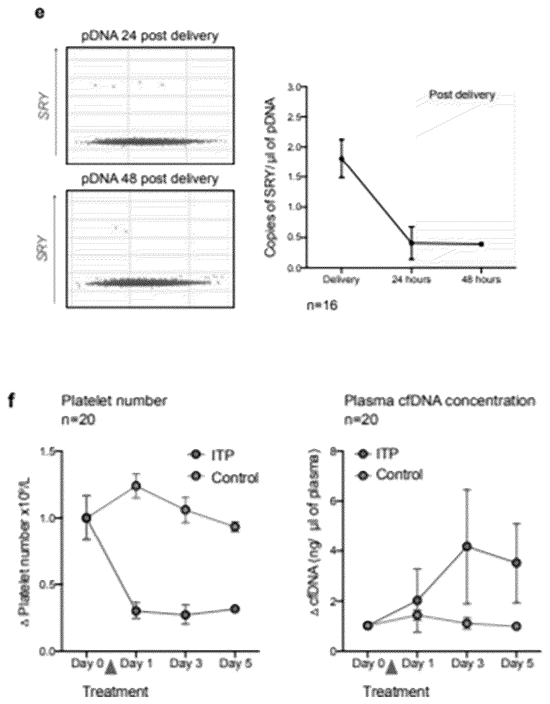

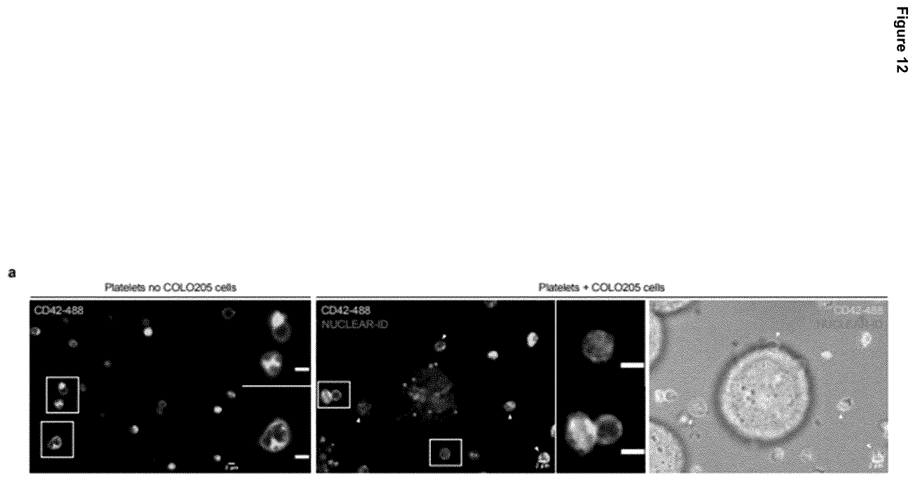

To determine whether DNA was detectable within platelets isolated from peripheral blood of human donors, we labelled platelets with NUCLEAR-ID Red DNA, a highly specific, cell permeable dye that intercalates with double stranded DNA. A proportion of platelets identified by their positivity for the platelet cell surface integrin CD42b showed a clear fluorescent signal for the DNA probe (FIG. 11a). To compare the relative abundance of DNA in platelets (pDNA) to that in platelet-depleted plasma, a protocol was developed to simultaneously isolate purified platelets and cfDNA from the same blood samples by sequential centrifugation (FIG. 11b). A cohort of 64 donors was studied, including individuals undergoing colonoscopy for cancer surveillance; patients with inflammatory bowel disease (Crohn's disease or ulcerative colitis) (n=43), and patients with known gastrointestinal cancers (n=21, including colorectal [CRC], oesophageal and pancreatic cancer). DNA was obtained from the platelet pellet in all cases, and in the majority of samples (55/64, 86%), more DNA (by 6.4+7.4 fold in genomic copies) was obtained from the platelet pellet than platelet-depleted plasma (cfDNA, P<0.0001, FIG. 11c). High purity of platelet pellets was confirmed by flow cytometry analysis and cytospin preparations, confirming that less than 2 in every 10,000 cells were non-platelet cell types.

To determine whether the DNA detected in platelets was captured during peripheral circulation or solely derived from parent megakaryocytes, we collected antenatal blood samples and looked for fragments of the Y-chromosome in the platelets of mothers carrying male babies. Using a Y-chromosome fluorescence in-situ hybridization (FISH) probe and droplet digital PCR (ddPCR), Y-chromosome fragments were visualized in the platelets of mothers carrying a male baby (FIG. 11d). In 100% (10/10) of samples, we correctly ascertained the sex of the fetus by the presence or absence of SRY alleles in maternal platelets using ddPCR. SRY remained detectable in platelets at 24 and 48 hours after delivery of the baby (FIG. 11e) and was not detected in mononuclear cells (FIG. 1d) or red blood cells from the same blood samples, indicating that the sequestration of free fetal DNA by blood cells is specific to platelets and not due to ‘contaminating’ cfDNA in the platelet pellet.

We reasoned that if platelets were clearing cfDNA from plasma, then the abundance of cell/platelet-free DNA in plasma would substantially increase if the platelet count was reduced. To test this, we induced acute and specific depletion of platelets in mice by treatment with an anti-CD42b anti-platelet antibody. In accordance with our hypothesis, the induction of immune thrombocytopenia led to an acute rise in cfDNA in plasma (FIG. 11f). Together, these data support a role for platelets in sequestration and clearance of cfDNA from plasma.

### Example 2—Platelets Capture DNA from Nucleated Cells

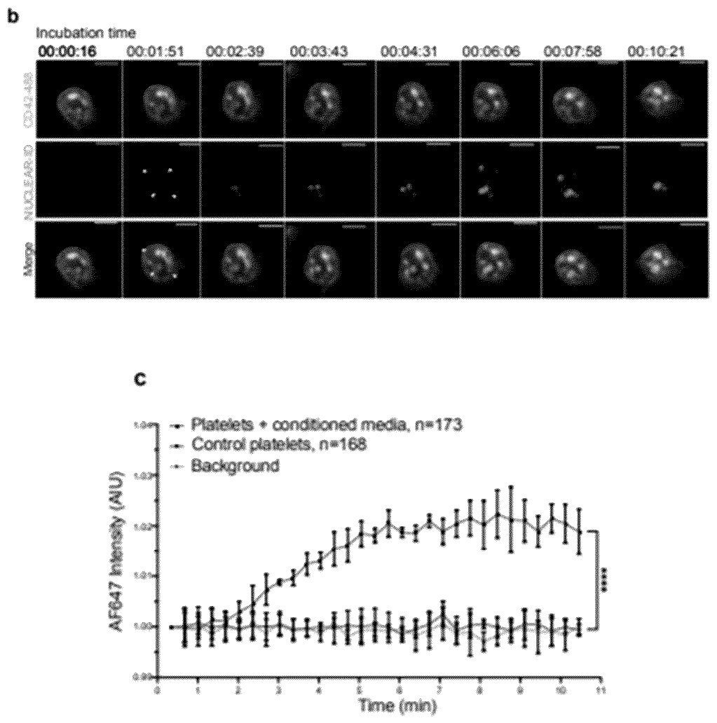

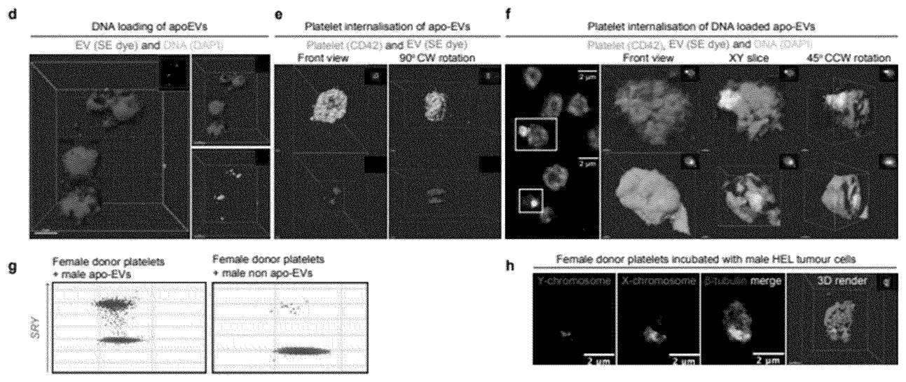

To confirm that platelets sequester chromosomal fragments released from nucleated cells, we used live cell imaging to visualize DNA uptake in vitro. Colorectal adenocarcinoma cells (COLO205) were labelled with cell-permeable, fluorescent DNA probes that either irreversibly intercalate with double stranded DNA (NUCLEAR ID) or covalently bind DNA (SIR-DNA), and washed 2× to remove any non-internalized probe prior to co-incubation with platelets. Acquisition of COLO205 cell-derived, labelled DNA by platelets was observed (FIG. 12a). To evaluate the time course of DNA capture, platelets were added to media conditioned by DNA-labelled COLO205 cells and platelet uptake of fluorescently-tagged DNA was measured at 16 second intervals over a 10 minute time course. DNA was rapidly visible in platelets within minutes (FIG. 12b), and the average fluorescence intensity of the platelets imaged plateaued at ˜6 minutes, suggestive of possible saturation of DNA uptake (FIG. 12c). Similar results were observed when the fluorescence signal of randomly selected individual platelets was tracked over time, following addition of platelets to COLO205-conditioned media. Together, these data reveal that DNA fragments released by nucleated cells are internalized by platelets, and that uptake is not dependent on physical contact between platelets and nucleated cells.

### Example 3—Nucleic Acid Transfer Via DNA-Loaded EVs

One mechanism by which cells remove excess cytoplasmic DNA created during aberrant cycles of mitosis and chromosomal instability is via the release of DNA-loaded extracellular vesicles (EVs), and EVs are also released during cellular apoptosis. Platelets can internalize EVs via their open canalicular system (OCS), and platelet uptake of tumour cell-derived EVs has previously been reported to mediate transfer of cancer cell-derived protein cargo and mRNA transcripts. However, whether EVs mediate transfer of DNA fragments from nucleated cells to platelets has not previously been explored. To determine whether EVs are mediators of DNA transfer to platelets, and the role of tumour cell apoptosis in DNA release, platelets were isolated from female donors and incubated for 3 hours with EVs isolated from BL2 cells—a human B-cell lymphoma cell line originating from a male donor or isogenic BL2 cells engineered to express the apoptosis-suppressing gene Bcl-2 (BL2-Bcl-2 cells). EVs were isolated from BL2 and BL2-Bcl-2 cells treated with UV irradiation to generate apoptotic EVs (apo-EVs from BL2 cells) and non-apoptotic (non-apo EVs) from BL2-Bcl-2 cells, and treated with DNase to remove any non-internalized DNA. Imaging of EVs confirmed internalized DNA within EVs (FIG. 12d), and DNA-loaded EVs were visible adherent to and within platelets following co-incubation (FIGS. 12e & 12f). To track DNA transfer more specifically, ddPCR was used to quantify SRY alleles. Copies of SRY were detectable in platelets from female donors only following incubation with BL2-derived EVs, and in significantly higher abundance following co-incubation with apo-EVs than with non-apo EVs (FIG. 12g,). Similarly, using whole chromosome X and Y FISH probes, fragments of both X- and Y-chromosomes were visualized in a subfraction of female platelets following co-incubation with HEL cells (FIG. 12h).

### Example 4—Detecting Cancer Mutations in Platelets

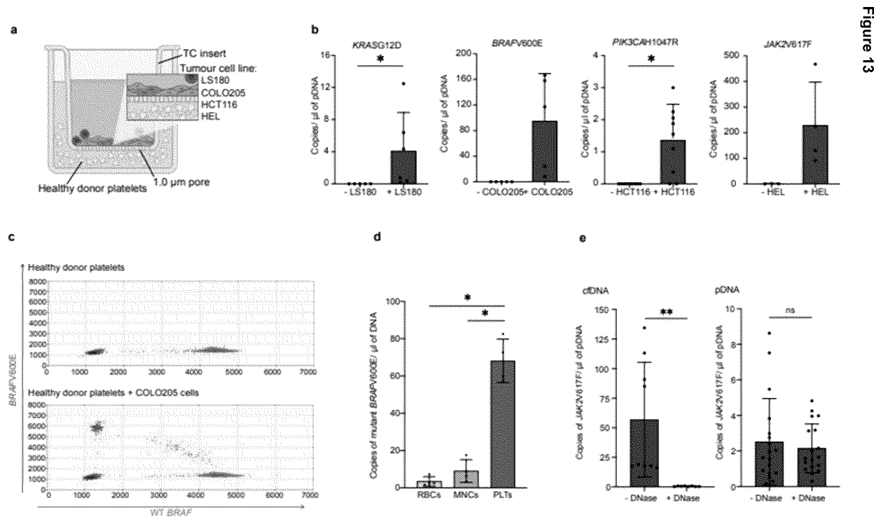

We next sought to determine whether DNA fragments bearing specific cancer-associated gene mutations could be detected in platelets. Platelets from healthy donors were incubated with a variety of human cancer cell lines harbouring common cancer-associated gene mutations, including COLO205, HCT116 and LS180 colorectal cells and HEL erythroleukemia cells (FIG. 13a). Cancer cell lines were separated from platelets by inserts with 1 μm pores that facilitated exchange of biomolecules and small EVs between compartments but prevented nucleated cells from entering the lower compartment. ddPCR of DNA isolated from platelets following co-incubation with cancer cell lines detected all relevant oncogenic mutations, including KRASG12D, BRAFV600E, PIK3CAH1047R and JAK2V617F (FIGS. 13b and 13c). No cancer-specific gene mutations were identified in healthy donor platelets incubated alone, but wild-type alleles were detected as expected (FIG. 13c), likely indicative of sequestration of DNA from non-transformed cells encountered by platelets during circulation, prior to blood sampling. To determine whether the ability to capture extracellular DNA was unique to platelets or shared by other blood cell types, we tested whether BRAFV600E derived from COLO205 cells was sequestered by platelets, peripheral blood mononuclear cells (MNCs) and red blood cells (RBCs) following addition of these cells to media conditioned for 12 hours by COLO205 cells. BRAFV600E was readily detectable in pDNA following incubation of platelets in COLO205-conditioned media as well as following direct platelet-COLO205 cell co-culture (FIG. 13d), whereas very few copies of mutant BRAF were detected in MNCs (p=0.0152) and RBCs (p=0.0022) (FIG. 13d), indicating substantially higher efficiency of detection of tumour cell-derived DNA in platelets than in other blood cell types, and also confirming that the mutant isoforms detected in platelets were not due to ‘contaminating’ residual tumour cell-conditioned media.

### Example 5—pDNA Is Protected from Nucleases

Our imaging data (FIG. 12a-c) suggested that pDNA was largely internalized, rather than adherent to the outer membrane. To confirm this, and to test whether extracellular DNA sequestered by platelets is protected by the platelet membrane from external nuclease digestion, conditioned media and platelets were treated with DNase following conditioning by HEL cells that carry the JAK2V617F mutation. The detection of JAK2V617F in HEL cell conditioned media was completely abrogated by DNase treatment (FIG. 13e). In contrast, there was no reduction in the abundance of JAK2V617F in platelets following DNase treatment (FIG. 13e), indicating that the majority of DNA is encapsulated by the platelet outer membrane and protected from enzymatic degradation.

### Example 6—Cellular Origin of pDNA Fragments

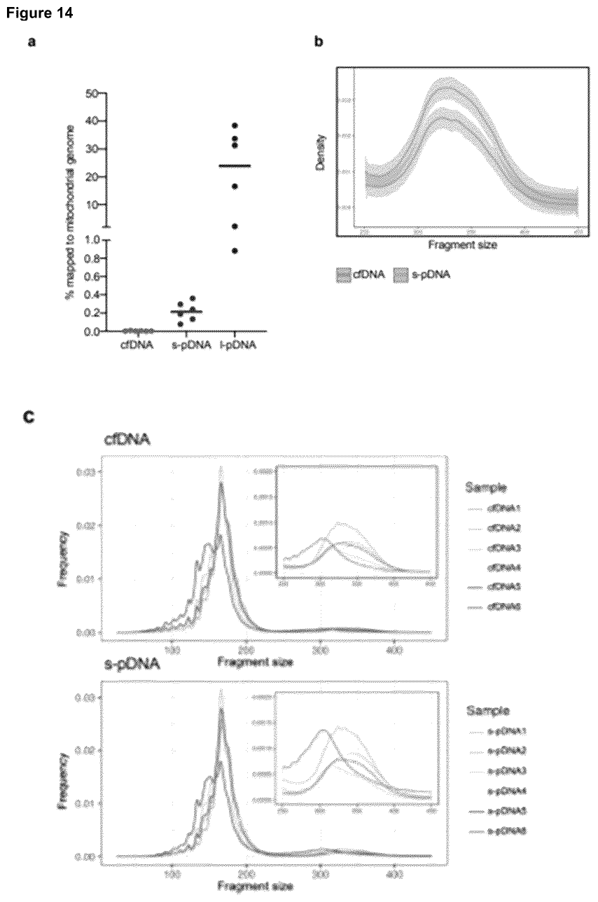

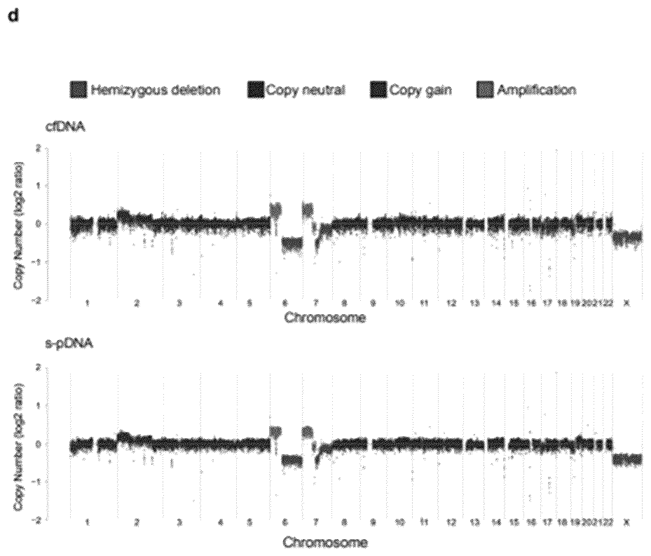

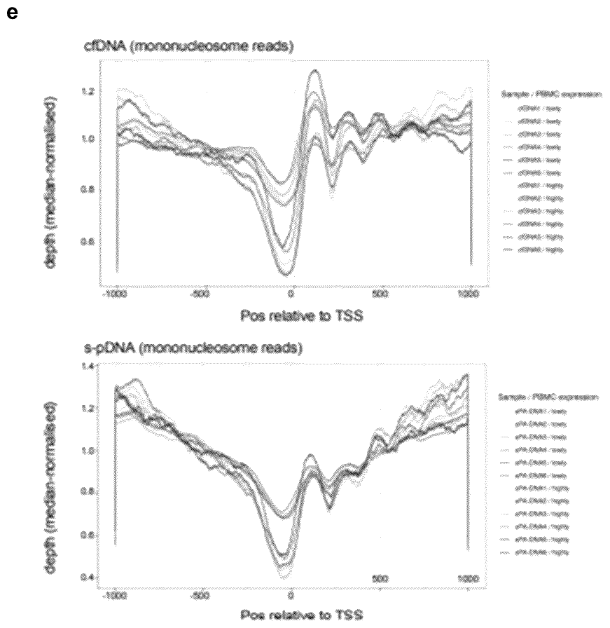

We next sought to confirm the cellular origins and genomic distribution of platelet-derived DNA. Electrophoresis analysis showed that pDNA contained fragments around 12,000-16,000 base pairs (bp), and a proportion of platelet samples also contained shorter fragments of around 120-160 bp, with a periodicity reflecting nucleosome footprints, similar to the fragmentation profile that is classically observed in cfDNA17. We selected 6 samples that contained both shorter and longer fragment length peaks from donors with a recent diagnosis of gastrointestinal cancer and performed whole genome sequencing (WGS) on pDNA and cfDNA isolated simultaneously from the same peripheral blood sample. Size selection was performed on the pDNA to separate shorter (less than 600 bp, “s-pDNA”) from longer fragments (“I-pDNA”). We first performed low pass WGS (mean target coverage of 1x) on s-pDNA, I-pDNA and paired cfDNA to determine cellular origin. Platelets contain a small number of mitochondria, estimated at 4-6 mitochondria per cells. As the mitochondrial genome is ˜16,500 bp, we reasoned that I-pDNA fragments might represent mitochondrial DNA. Indeed, a significantly higher subfraction (0.9-38%) of I-pDNA mapped to the mitochondrial genome, compared to <0.001-0.36% of the shorter fragments (FIG. 14a). However, the majority of fragments from both s- and I-pDNA mapped to the nuclear genome (98.6-99.9% for s-pDNA and 62-99.1% for I-pDNA, FIG. 14a)

### Example 7—Genomic Coverage of pDNA Mirrors cfDNA

We then obtained deeper sequencing (mean sequencing depth 15x±3/12x±1 for cfDNA and s-pDNA, respectively) of the paired cfDNA and s-pDNA. No notable differences were detected in sequencing quality (base quality, 3′ bias, mapping rate) between s-pDNA and cfDNA, except for a higher estimated PCR duplication rate (cfDNA: 3.2%±0.6%, pDNA: 11.3%±7.2%), indicating lower complexity of short pDNA libraries, presumably as a result of size selection.

Mapping across the genome revealed that the fragments in platelets covered the entire nuclear genome, mirroring that seen in cfDNA and demonstrating that sampling of pDNA would enable oncogenic variations in genes across the chromosomes to be detected. Compared to cfDNA, the s-pDNA fragments showed more even coverage, as indicated by a significantly lower Gini index (0.22±0.03 for s-pDNA vs. 0.27±0.03 for cfDNA).

Nucleosome-bound cfDNA is more protected from degradation than nucleosome-free DNA. This leads to a characteristic fragment-length distribution of cfDNA, with mono- and di-nucleosome length peaks at approx. 167 bp and 320 bp. The peak mean fragment length for s-pDNA was 166 bp±0.8, with a second, di-nucleosome peak at approximately 328 bp. In all 6 cases sequenced, the dinucleotide peak was fractionally but significantly larger in s-pDNA than cfDNA (FIG. 14b). One explanation for this may be the increased protection of DNA fragments in platelets from nuclease digestion, as observed in our in vitro experiments (FIG. 13e).

Several studies have shown that cfDNA fragments of tumour cell origin are of shorter length than wild-type fragments. In accordance with this, we observed that both the mono- and the di-nucleotide fragment length were notably shorter in both pDNA and cfDNA in sample 6, derived from a patient with an inoperable pancreatic tumour, than in the other 5 samples from individuals who had treatment with surgery and/or chemo-irradiation prior to blood sampling, indicating that fragmentomic analysis may be applied to platelet DNA to detect the presence of cancer (FIG. 14c).

To confirm the capture of circulating tumour cell-derived DNA (ctDNA) by platelets, we used iChorCNA to detect copy number alterations (CNA) and estimate the fraction of tumour-derived DNA within the samples. Clonal amplifications and deletions in chromosomes 6 and 7 as well as sub-clonal amplifications in chromosome 2 were detected in both cfDNA and spDNA from sample 6, with an estimated tumour fraction of 25% (FIG. 14d). No CNAs were seen in either cfDNA or s-pDNA in the other 5 samples. CNAs detected in s-pDNA mirrored those present in cfDNA, and the estimated proportion of tumour-derived DNA was highly correlated between s-pDNA and cfDNA. Selecting DNA fragments smaller than 150 bp increased the estimated tumour content of s-pDNA from 25% to 30%, in parallel to the increase seen in cfDNA. Notably, no CNAs were detected in the I-pDNA from sample 6, in line with previous reports that ctDNA is enriched in shorter fragments and indicating that the large and small fragments of pDNA may have different cell and/or tissue origins.

The enrichment for nucleosome-bound DNA in cfDNA has previously been used to gain insight into gene expression patterns and cell-of-origin, with prior reports showing that a depletion of reads occurs over transcriptional start sites (TSS) with periodicity in coverage relative to the TSS. In addition, DNA methylation studies have shown that the majority (>90%) of cfDNA in healthy individuals is of haematopoietic cell origin including from neutrophils, monocytes, erythroblasts and megakaryocytes, with a small contribution from vascular endothelial cells and hepatocytes. We found very similar depletion in sequencing coverage and periodicity around TSS for genes that are highly expressed in peripheral blood mononuclear cells (PBMNCs) for s-pDNA and cfDNA, to that previously reported for cfDNA, suggesting a shared cellular origin of pDNA and cfDNA (FIG. 14e). Notably, the depletion in coverage for high-expression PBMNC genes was less marked for Sample 6 for which the estimated tumour fraction was 25% than for the other samples (FIG. 14e).

### Example 8—KRASG12D Alleles More Abundant in Platelets than cfDNA in CRC Mice

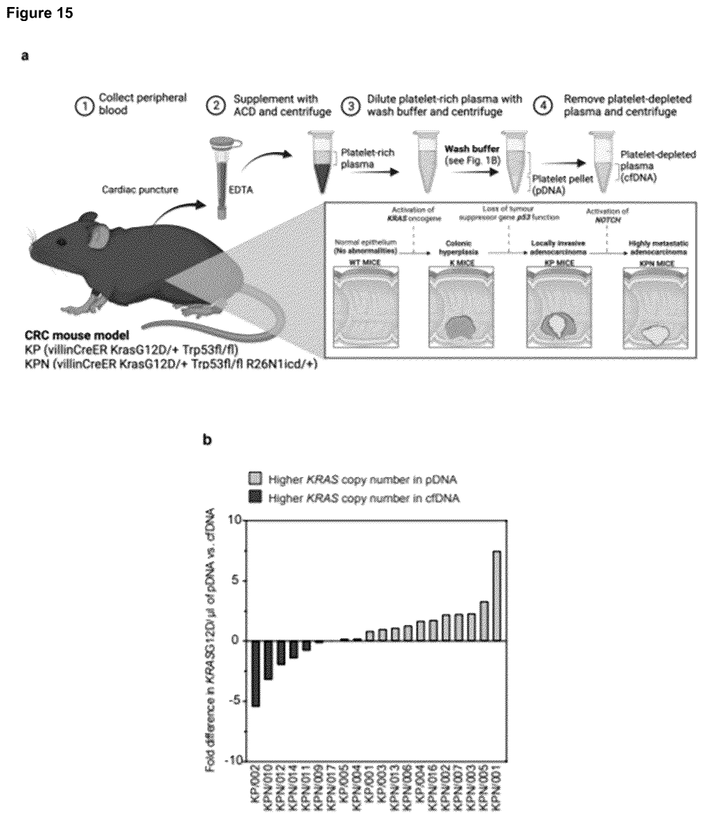

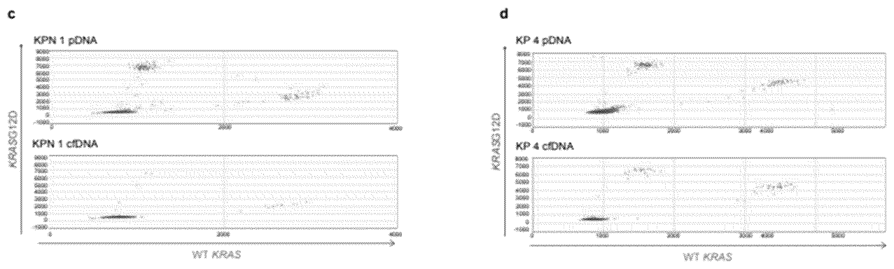

Having demonstrated that platelets contain tumour cell-derived DNA, we explored the utility of this phenomenon for the detection of cancer gene aberrations as a liquid biopsy approach in vivo, hypothesizing that analysis of pDNA might be useful for cancer screening. We used a mouse model of colorectal carcinoma in which expression of KRASG12D and TP53 (KP) mutations are induced via the villin promoter, resulting in locally-invasive colorectal adenocarcinoma with a low incidence of metastases, and mice with KRASG12D/+, TP53fl/fl and NOTCHfl/+ (KPN), with more aggressive and metastatic disease (FIG. 15a). To determine therelative sensitivity of cfDNA and pDNA for the detection of tumour-derived mutant alleles, pDNA and cfDNA were extracted simultaneously from the same blood sample (FIG. 15a), and ddPCR was used to detect mutant KRASG12D alleles. Similar to our observation in human peripheral blood, DNA was in higher abundance in platelets than in platelet-depleted plasma in murine samples. KRASG12D was readily detectable in mice in both cfDNA and platelets of mice with colorectal carcinoma. Remarkably, in just over half of the mice (13/20, 65%), copies of KRASG12D per μL of DNA were in higher abundance in pDNA than in cfDNA, both in mice with locally invasive (KP) and metastatic (KPN) disease (FIGS. 15b-15d). These data suggest that including platelet DNA analysis in liquid biopsy methods may improve the sensitivity of cancer screening in many cases, and that current liquid biopsy approaches using only platelet-poor plasma for analysis of cfDNA are missing substantial genetic information that is contained within platelets.

### Example 9—BRAFV600E In Platelets of Patients with Pre-Cancer

Improving the sensitivity for liquid biopsy screening is particularly crucial in the setting of low tumour-burden disease and in patients with pre-malignant lesions, where the abundance of ctDNA is low. Sessile serrated lesions (SSLs) are high-risk, pre-malignant colon polyps that account for around one quarter of colorectal cancers. The majority (>75%) are driven by mutations in BRAF, typically BRAFV600E, and detection of BRAFV600E in cfDNA has been investigated as an SSL screening tool, with high specificity (100%) albeit low sensitivity (16.9%). To explore the utility of pDNA for detecting pre-malignant lesions, we collected platelets and cfDNA from patients found to have SSLs at colonoscopy (n=29). Control samples (n=14) were collected from patients with inflammatory bowel disease and from those invited for colonoscopy through the U.K. Bowel Cancer Screening Program in whom no polyps or cancerous lesions were detected.

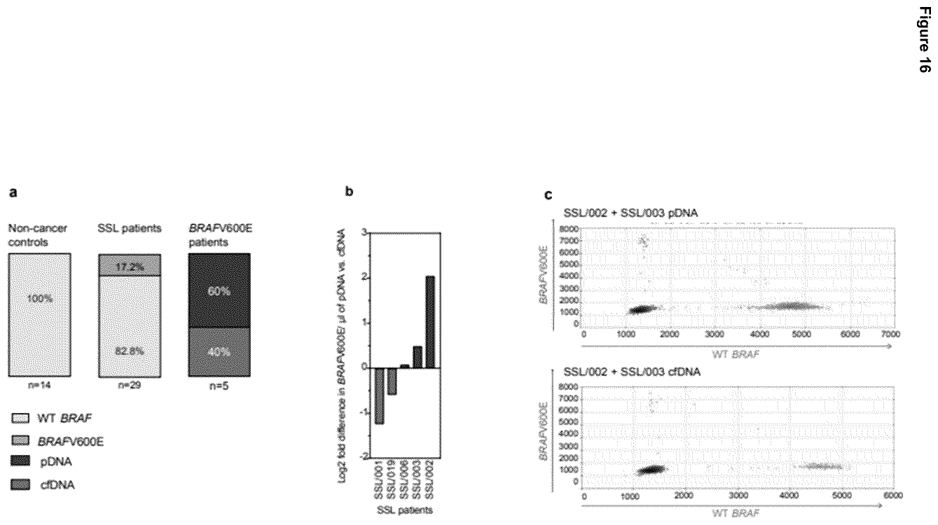

Using ddPCR, BRAFV600E was detected in either the cfDNA and/or pDNA in 17.2% of the patients with serrated polyps (5/29, FIG. 16a), a detection rate consistent with previous reports for the frequency of detectable ctDNA in this patient cohort. Mirroring our findings in the mouse model, around half (3/5) of the patients with detectable ctDNA had a higher number of copies of BRAFV600E in pDNA than in cfDNA (FIGS. 16b & 16c). BRAFV600E was detected in none of the pDNA samples from 14 controls (FIG. 16a), demonstrating high specificity of targeted mutational analysis of platelet DNA in this setting. Collectively, these data reveal a new biological role for platelets in sequestration of cfDNA from plasma, and indicate that analysis of pDNA may be useful to detect genetic aberrations in a number of clinical settings.

### DISCUSSION

Genomic material is continuously shed into human body fluids through cell death, aberrant mitotic cycles or regulated DNA extrusion. Release of cfDNA is increased in malignancy, inflammation and following tissue damage, and several physiological mechanisms exist to limit its abundance, as excess extrachromosomal DNA in the cytosol and in plasma is proinflammatory. Similar to red blood cells, platelets express nucleic acid sensing receptors and their capture of pathogen-derived nucleic acids is an important component of innate immunity. Here, we present data that indicates a role for platelets that was previously unappreciated—as scavengers of endogenous extracellular cfDNA, including tumour-derived and free fetal DNA. Detection and analysis of cfDNA is rapidly being implemented in several clinical settings including cancer screening, molecular profiling and monitoring of treatment responses as well as in prenatal diagnosis. Such liquid biopsy approaches are minimally invasive, enable access to tumours in difficult-to-reach biopsy sites and more reliably capture intra- and inter-tumour heterogeneity than traditional tissue sampling. Currently, a major limitation in the setting of cancer diagnostics is the poor sensitivity for low tumour burden disease, and pre-analytical approaches to increase capture of ctDNA would be of significant interest. Our data suggest that a substantial proportion of ctDNA is contained within platelets, and is currently being missed by standard sampling protocols that extract cfDNA from platelet-depleted plasma. In addition, we show that pDNA is more protected from nuclease digestion than cfDNA external to platelets. Platelets have long been associated with carcinogenesis and metastasis, contributing to tumour angiogenesis, tumour cell extravasation and the shielding of metastasizing cancer cells from immunosurveillance. Prior studies have also highlighted a role for platelets in cancer diagnostics. The unique biophysical properties of platelets include a surface connected, open canalicular system that enables rapid transport of molecules into and out of the platelet interior, such as the internalization of extracellular vesicles containing a cargo of tumour biomarkers. Platelets are highly abundant and easy to isolate, and therefore ideally poised as ‘sentinels’ for genetic perturbations in tissues and for use in liquid biopsy approaches. Studies on platelet nucleic acids have previously shown that the platelet transcriptome is altered in the presence of cancer by altered splicing of the platelet mRNA repertoire that derives from the parent megakaryocytes, as well as by ingestion of mRNA transcripts from tumour cells. However, mRNA is less stable than DNA, and the platelet transcriptome and spliceosome is also altered in non-malignant pathologies, therefore likely to be less specific than screening for oncogenic aberrations in platelet DNA. We have shown that pDNA contains similar information to standard cfDNA isolated from platelet-poor plasma but not directly tested whether combined analysis of cfDNA plus pDNA increases the sensitivity of screening methods, nor if integration of DNA with mRNA signatures or protein cargo in platelets may be of added value. We have also not yet explored whether some of the DNA cargo in platelets originates from their parent megakaryocytes. Nonetheless, collectively, our data highlights a novel aspect of platelet biology and demonstrates the utility of pDNA analysis for liquid biopsy genetic screening in multiple clinical settings. This study paves the way for future research to further establish a role for platelets in cfDNA clearance and homeostasis, and the utility of this phenomenon in clinical diagnostic settings.

1. A method for the detection or prognosis of clonal haematopoiesis comprising:
providing a biological sample comprising thrombocytes;
extracting nucleic acid from said biological sample;
analysing said nucleic acid to identify the presence of one or more clonal haematopoiesis associated mutations; and
indicating the presence or prognosis of clonal haematopoiesis based on the presence of one or more clonal haematopoiesis associated mutations.

2. The method according to claim 1, wherein the one or more clonal haematopoiesis associated mutations are selected from JAK2^{V617F}, JAK2 exon12, CALR exon9, MPL^{S5050}, MPL^{W515}, CBL exon 8, CBL exon 9, KRAS exon 2, KRAS exon 3, GNB1 exon 5, DNMT3A all exons, TET2 all exons, ASXL1 exon 12, IDH2 exon 4, SF3B1 exon 14, SF3B1 exon 15, SRSF2^{P95}, U2AF1 exon 2, U2AF1 exon 6, PPM1D exon 6, TP53 all exons.

3. The method according to claim 1 or claim 2, wherein the method comprises extracting DNA and/or RNA from said biological sample.

4. The method according to claim 3 wherein, the method comprises a step of converting said RNA into cDNA.

5. The method according to any preceding claim, wherein the method further comprises:
providing a biological sample comprising granulocytes;
extracting nucleic acid from said biological sample; and
analysing said nucleic acid to identify the presence of one or more clonal haematopoiesis associated mutations.

6. The method according to claim 4 or claim 5, wherein the RNA is converted to cDNA via reverse transcription.

7. The method according to any preceding claim, wherein a subject that is diagnosed which clonal haematopoiesis is selected for further monitoring.

8. The method according to any preceding claim, wherein a subject that is diagnosed which clonal haematopoiesis is identified as being at high risk of cardiovascular disease, heart failure, diabetes, autoimmune disease and/or myeloid blood cancers.

9. The method of claim any preceding claim, wherein the presence of one or more clonal haematopoiesis associated mutations is identified via droplet digital PCR, next generation sequencing, allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), pyro-sequencing, HPLC (high-performance liquid chromatography) or oligonucleotide ligation assay (OLA).

10. A method for the detection or prognosis of cancer comprising:
providing a biological sample comprising thrombocytes;
extracting nucleic acid from said biological sample;
analysing said nucleic acid to identify the presence of one or more cancer associated nucleic acid fragments; and
indicating the presence or prognosis of cancer based on the presence of one or more cancer associated nucleic acid fragments.

11. The method according to claim 10, wherein the cancer associated nucleic acid fragment is DNA and/or RNA.

12. The method according to any one of claim 10 or 11, wherein the cancer associated nucleic acid fragment is a cell free nucleic acid fragment that has been released from a cancer and taken up by said thrombocytes.

13. The method according to any one of claims 10 to 12, wherein the cancer associated nucleic acid fragment comprises a cancer associated modification, or a cancer specific mutation, methylation pattern, genetic aberration and/or fragmentation pattern.

14. The method according to any one of claims 10 to 13, wherein the cancer associated nucleic acid fragment is selected from nucleic fragments comprising one or more of the following mutations BRAF^{V600E}, KRAS^{G12D}, PIKCAH^{1047R}, TP53^{R273H}.

15. The method according to any one of claims 10 to 14, wherein the cancer associated nucleic acid fragment is identified via droplet digital PCR, next generation sequencing, allele-specific polymerase chain reaction (PCR), high resolution melting curve analysis, genomic sequencing fluorescence in situ hybridization (FISH); comparative genomic hybridization (CGH), Restriction fragment length polymorphism RELP), amplification refractory mutation system (ARMS), reverse transcriptase PCR (RT-PCR), real-time PCR, multiplex ligation-dependent probe amplification (MLPA), denaturing gradient gel electrophoresis (DGGE), single strand conformational polymorphism (SSCP), chemical cleavage of mismatch (CCM), protein truncation test (PTT), or oligonucleotide ligation assay (OLA), methylation analysis, fragmentation pattern analysis.

16. The method according to any one of claims 10 to 15, wherein the cancer associated nucleic acid fragment comprises 10 to 1500 nucleotides.

17. The method according to any one of claims 10 to 16, wherein the cancer associated nucleic acid fragment is associated with a solid tumour.

18. The method according to any one of claims 10 to 17, wherein the cancer associated nucleic acid fragment is associated with sarcoma, carcinoma, and/or lymphoma.

19. The method according to any one of claims 10 to 18, wherein the cancer associated nucleic acid fragment is associated with gastric cancer, lung cancer, non-small cell lung cancer, lung adenocarcinoma, lung squamous cell carcinoma, bone cancer, pancreatic cancer, colon cancer, colorectal cancer, skin cancer, cancer of the head or neck, head and neck squamous cell carcinoma, melanoma, uterine cancer, ovarian cancer, rectal cancer, cancer of the anal region, stomach cancer, testicular cancer, breast cancer, brain cancer, hepatocellular cancer, carcinoma of the fallopian tubes, carcinoma of the endometrium, carcinoma of the cervix, carcinoma of the vagina, carcinoma of the vulva, cancer of the oesophagus, cancer of the small intestine, cancer of the endocrine system, cancer of the thyroid gland, cancer of the parathyroid gland, cancer of the adrenal gland, kidney cancer, sarcoma of soft tissue, cancer of the urethra, cancer of the bladder, renal cancer, thymoma, urothelial carcinoma leukaemia, prostate cancer, prostatic adenocarcinoma mesothelioma, adrenocortical carcinoma, lymphomas, such as such as Hodgkin's disease, non-Hodgkin's, and multiple myelomas.

20. A method of determining a treatment for a subject, comprising:
the method of detection or prognosis according to any one of claims 10 to 19; and
determining a suitable treatment.

21. The method according to any preceding claim wherein the biological sample is processed to provide an isolated sample of thrombocytes.

22. The method according to any preceding claim wherein the biological sample is a blood sample.

23. A kit comprising reagents for the extraction of nucleic acid from platelets and a panel of reagents that specifically bind to and/or amplify one or more clonal haematopoiesis associated mutations, and optionally instructions for use.

24. The kit according to claim 23, wherein the reagents are for the parallel analysis of cDNA and gDNA comprising one or more clonal haematopoiesis or cancer associated mutations.

25. The kit according to claim 23 or 24, wherein the reagents are for the detection of clonal haematopoiesis associated mutations in one or more of JACK2, CALR, MPL, CBL, KRAS, GNB1, DNMT3A, TET2, ASXL1, IDH2, SF3B1, SRSF2, U2AF1, PPM1D, TP53.

26. A kit comprising reagents for the extraction of nucleic acid from platelets and a panel of reagents that specifically bind to and/or amplify one or more cancer associated modifications, or cancer specific mutations.

27. A method of treatment of a subject with cancer comprising the steps of:
providing a biological sample comprising thrombocytes,
extracting nucleic acid from said biological sample,
analysing said nucleic acid to identify the presence of one or more cancer associated nucleic acid fragments,
selecting a treatment; and
administering the treatment.

28. A method of preparing a nucleic acid fraction comprising the steps of:
providing a biological sample comprising thrombocytes,
extracting nucleic acid from said biological sample to form a nucleic acid sample, enriching said nucleic acid sample for one or more cancer associated nucleic acid fragments and/or clonal haematopoesis associated fragments.

29. A method for antenatal screening, comprising the steps of:
providing a biological sample comprising thrombocytes, obtained from a pregnant woman;
extracting nucleic acid from said biological sample;
analysing said nucleic acid for genetic information related to the foetus.
